(19) United States
(12) Patent Application Publication
(10) Pub. No.: US 2005/0143420 A1
(43) Pub. Date: Jun. 30, 2005

(54) Methods and compositions for the treatment and management of hemoglobinopathy and anemia

(76) Inventors: Laure Moutouh-de Parseval, San Diego, CA (US); Kyle W.H. Chan, San Diego, CA (US); Helen Brady, San Diego, CA (US)

Correspondence Address: JONES DAY, 222 EAST 41ST ST, NEW YORK, NY, 10017, US

(21) Appl. No.: 11/004,736

(22) Filed: Dec. 2, 2004

Related U.S. Application Data
(60) Provisional application No. 60/526,910, filed on Dec. 2, 2003.

(51) Int. Cl. A61K 31/454

(52) U.S. Cl. 514/323

(57) ABSTRACT

The present invention is directed to the use of immunomodulatory compounds, particularly members of the class of compounds known as IMiDs™, and more specifically the compounds 4-(Amino)-2-(2,6-dioxo(3-piperidyl))-isoindoline-1,3-dione and 3-(4-amino-1-oxo-1,3-dihydroisoindol-2-yl)-piperidine-2,6-dione, to induce the expression of fetal hemoglobin genes, genes essential for erythropoiesis, and genes encoding alpha hemoglobin stabilizing protein, within a population of CD34⁺ cells. These compounds are used to treat hemoglobinopathies such as sickle cell anemia or β-thalassemia, or anemias caused by disease, surgery, accident, or the introduction or ingestion of toxins, poisons or drugs.

This application claims benefit of U.S. Provisional Application Ser. No. 60/526,910 filed Dec. 2, 2003, which is hereby incorporated by reference in its entirety.

### 1. FIELD OF THE INVENTION

This invention is directed to methods of treating, preventing and/or managing hemoglobinopathies, such as sickle cell anemia, and other anemias, such as disease- or drug-induced anemias, by administration of members of the class of thalidomide analogs known as IMiDs™, particularly the IMiDs™ 4-(Amino)-2-(2,6-dioxo(3-piperidyl))-isoindoline-1,3-dione (also known as a-(3-aminophthalimido) glutarimide; Celgene Corporation) and 3-(4-amino-1-oxo-1,3-dihydroisoindol-2-yl)-piperidine-2,6-dione (also known as 3-(4′aminoisoindoline-1′-one)-1-piperidine-2,6-dione; Celgene Corporation), and pharmaceutical compositions comprising such compounds.

### 2. BACKGROUND OF THE INVENTION

2.1. Sickel Cell Anemia and Other Hemoglobinopathies

Sickle cell anemia (“SCA”) is a genetic hemolytic anemia associated with abnormal hemoglobins, designated hemoglobin S. The disease is reported to be caused by a decreased electrical charge in hemoglobin S due to an amino acid substitution, which in turn results in lower solubility of the substituted hemoglobin S. The Merck Manual of Diagnosis and Therapy, 17^{th} Ed., Merck Research Laboratories, Whitehouse Station, N.J., page 878 (1999). The less soluble hemoglobin S forms a semi-solid gel of rod-like tactoids that causes red blood cells to assume a crescent, sickle-like shape. These distorted and inflexible red blood cells adhere to vascular endothelium and plug small arterioles and capillaries, which leads to occlusion and infarction. As the sickled red blood cells are too fragile to withstand the mechanical pressure of blood circulation, hemolysis occurs when they enter the circulation.

SCA is generally associated with a specific ethnic group, i.e., African-Americans and other persons descended from tropical sub-Saharan African populations. The patients suffer acute pain caused by the occlusion caused by the sickled red blood cells. The life span of the sickled red blood cells is approximately two weeks, whereas the average life span of normal red blood cells is about four months. This shortened life span in turn leads to chronic anemia.

The symptoms of SCA include impairment of growth and development; increased susceptibility to infections; a tower-shaped skull; bone changes such as cortical thinning, irregular bone densities and new bone formation within the medullary canal; small spleens due to autosplenectomy; increased chance of rheumatic or congenial heart diseases; progressive decrease of lung and kidney finction; and acute chest syndrome. Acute chest syndrome is the major cause of death, and is characterized by sudden onset of fever, chest pain, leukocytosis and pulmonary parenchymal infiltrates on chest x-ray.

Current approaches to the treatment of SCA include the induction of fetal hemoglobin, relaxation of blood vessels, the reduction of erythrocyte adhesion, and the use of Gardos channel antagonists. Iyamu and Asakura, Expert Opin. Ther. Patents, 13(6):807-813 (2003). The Gardos channel is a calcium-activated potassium channel described by Gardos (Curr. Top. Membr. Transp. 10:217-277 (1978) and Nature London 279:248-250 (1979)).

The most studied and used SCA treatment is the oral administration of hydroxyurea (HU). HU is believed to exert its effect by inducing the production of fetal hemoglobin (HbF). HU, however, is not effective in all patients; some patients fail to respond at all to HU, while others experience myelosuppression. Iyamu and Asakura, supra. SCA has also been treated with a natural herbal extract known as HEMOXIN™ (formerly designated NIPRISAN™), which appears to exert its anti-sickling effect by covalently binding to HbS. See U.S. Pat. No. 5,800,819. Iyamu and Asakura, supra. HEMOXIN™ is not yet FDA-approved for use in the treatment of SCA. One group is currently exploring the use of clotrimazole and other Gardos channel blockers in an effort to reduce the dehydration characteristic of sickled erythrocytes. Iyamu and Asakura, supra. The efficacy of such compounds, however has not been demonstrated. Other SCA treatments include intravenous solutions of glucose and electrolytes, narcotic analgesics, and transfusion for extremely severe cases of anemia. Given the nascent state of the majority of SCA therapeutics, a safer and effective therapy is needed for the treatment and management of SCA.

Treatments that increase the production of fetal hemoglobins are attractive because they increase the amount of total hemoglobin available to an individual suffering from a hemoglobinopathy or from anemia. In the adult, two types of hemoglobin, hemoglobin α and hemoglobin β, predominate, almost to the exclusion of other hemoglobin types. In contrast, two additional hemoglobins, hemoglobin ε and hemoglobin γ, are present in the fetus. Hemoglobin ε is a predominant form in early development, but ceases to be present in the fetus by approximately eight weeks of development. Hemoglobin γ, in contrast, is present early in development, reaching a peak percentage of total hemoglobin, of about 45%, at approximately 6-30 weeks gestation. It then diminishes in percentage of total hemoglobin from approximately 6 weeks prior to birth to approximately 40 weeks after birth. While present in an individual after 40 weeks of age, it constitutes less than 2% of the total hemoglobin present in the bloodstream thereafter.

2.2. IMIDS™

A class of compounds, referred to as IMiDS™ (Celgene Corporation) or Immunomodulatory Drugs, has been identified which show potent inhibition of TNFα and marked inhibition of LPS induced monocyte IL-1β and IL-12 production. LPS induced IL-6 is also inhibited by immunomodulatory compounds, albeit partially. These compounds are potent stimulators of LPS induced IL-10. IMiDs™ have been demonstrated to modulate the differentiation of CD34+ cells along myeloid and erythroid pathways. See U.S. Application Publication No. 2003/0235909, published Dec. 25, 2003, which is hereby incorporated herein in its entirety. Particular examples of IMiDs™ include, but are not limited to, the substituted 2-(2,6-dioxopiperidin-3-yl) phthalimides and substituted 2-(2,6-dioxopiperidin-3-yl)-1-oxoisoindoles described in U.S. Pat. Nos. 6,281,230 and 6,316,471, both to G. W. Muller, et al. IMiDs™ have not previously been identified as candidates for the treatment of hemoglobinopathies or anemia, or as modulators of genes involved in erythropoiesis.

### 3. SUMMARY OF THE INVENTION

The present invention is directed to methods of treating individuals having anemia or a hemoglobinopathy, comprising administering an effective amount of a compound of the invention. Thus, in one embodiment, the invention provides a method of treating an individual having anemia or a hemoglobinopathy, said method comprising administering to said individual an immunomodulatory compound, or a pharmaceutically acceptable salt, solvate, hydrate, stereoisomer, clathrate or prodrug thereof. In a specific embodiment, said anemia is an anemia induced by or related to the administration of a drug or chemotherapy. In another specific embodiment, said immunomodulatory compound is an amino-substituted thalidomide. In a more specific embodiment, said immunomodulatory compound is an IMiD™. In a more specific embodiment, said IMiD™ is α-(3-aminophthalimido) glutarimide (also known as 4-(Amino)-2-(2,6-dioxo(3-piperidyl))-isoindoline-1,3-dione); an analog or prodrug of α-(3-aminophthalimido) glutarimide; 3-(4′aminoisoindoline-1′-one)-1-piperidine-2,6-dione; an analog or prodrug of 3-(4′aminoisoindoline-1′-one)-1-piperidine-2,6-dione, or a compound of the formula

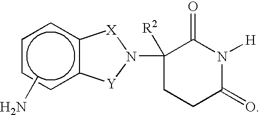

In another more specific embodiment, said IMiD is 1-oxo-2-(2,6-dioxopiperidin-3-yl)-5-aminoisoindoline, 1-oxo-2-(2,6-dioxopiperidin-3-yl)-4-aminoisoindoline, 1-oxo-2-(2,6-dioxopiperidin-3-yl)-6-aminoisoindoline, 1-oxo-2-(2,6-dioxopiperidin-3-yl)-5-aminoisoindoline, 1,3dioxo-2-(2,6-dioxopiperidin-3-yl)-4-aminoisoindoline, or 1,3dioxo-2-(2,6-dioxopiperidin-3-yl)-5-aminoisoindoline.

In another specific embodiment, the method of treatment additionally comprises treating said individual with a second compound, wherein said second compound is a compound that induces fetal hemoglobin, a compound that relaxes blood vessels, a compound that when covalently bound to hemoglobin S reduces the self-aggregation of hemoglobin S, a compound that is a Gardos channel antagonist, or a compound that reduces red blood cell adhesion. In a more specific embodiment, said second compound is hydroxyurea, a guanidino derivative, nitrous oxide, butyrate or a butyrate derivative, an aldehyde or an aldehyde derivative, a plant extract having antisickling activity (e.g., NIPRISAN™ (HEMOXIN™)), clotrimazole, a derivative of triarylmethane, a monoclonal antibody or a polyethylene glycol derivative.

In another specific embodiment, the method of treatment additionally comprises treating said individual with at least one cytokine. In a more specific embodiment, said at least one cytokine is erythropoietin (Epo), SCF, GM-CSF, Flt-3L, TNFα, IL-3, or any combination thereof. In another specific embodiment of the method, said individual is a mammal. In a more specific embodiment, said individual is a human.

In another embodiment, the invention provides a method of modulating the differentiation of a CD34⁺ stem or precursor cell to an erythroid lineage comprising differentiating said cell under suitable conditions and in the presence of an immunomodulatory compound, or a pharmaceutically acceptable salt, solvate, hydrate, stereoisomer, clathrate or prodrug thereof. In a more specific embodiment, said immunomodulatory compound is an amino-substituted thalidomide. In another more specific embodiment, said immunomodulatory compound is an IMiD. In an even more specific embodiment, said IMiD is α-(3-aminophthalimido) glutarimide (also known as 4-(Amino)-2-(2,6-dioxo(3-piperidyl))-isoindoline-1,3-dione); an analog or prodrug of α-(3-aminophthalimido) glutarimide; 3-(4′aminoisoindoline-1′-one)-1-piperidine-2,6-dione; an analog or prodrug of 3-(4′aminoisoindoline-1′-one)-1-piperidine-2,6-dione, or a compound of the formula 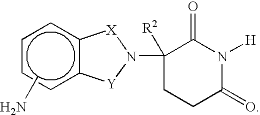
In another even more specific embodiment, said IMiD is 1-oxo-2-(2,6-dioxopiperidin-3-yl)-5-aminoisoindoline, 1-oxo-2-(2,6-dioxopiperidin-3-yl)-4-aminoisoindoline, 1-oxo-2-(2,6-dioxopiperidin-3-yl)-6-aminoisoindoline, 1-oxo-2-(2,6-dioxopiperidin-3-yl)-5-aminoisoindoline, 1,3dioxo-2-(2,6-dioxopiperidin-3-yl)-4-aminoisoindoline, and 1,3dioxo-2-(2,6-dioxopiperidin-3-yl)-5-aminoisoindoline. In another specific embodiment, said CD34⁺ stem or precursor cell is a cell in vitro. In another specific embodiment, said CD34⁺ stem or precursor cell is a cell in vivo.

In another specific embodiment, the method additionally comprises contacting said cell with at least one cytokine. In a more specific embodiment, said at least one cytokine is erythropoietin, SCF, GM-CSF, Flt-3L, TNFα, IL-3, or any combination thereof.

The present invention also provides pharmaceutical compositions comprising the compounds of the invention and another compound or cytokine. Thus, the invention provides a pharmaceutical composition comprising in a pharmaceutically-acceptable carrier an IMiD™ and a second compound, wherein said second compound is a compound that induces fetal hemoglobin, a compound that relaxes blood vessels, a compound that when covalently bound to hemoglobin S reduces the self-aggregation of hemoglobin S, a compound that is a Gardos channel antagonist, or a compound that reduces red blood cell adhesion. In a more specific embodiment, said second compound is hydroxyurea, a guanidino derivative, nitrous oxide, butyrate or a butyrate derivative, an aldehyde or an aldehyde derivative, a plant extract having antisickling activity (e.g, HEMOXIN™), clotrimazole, a derivative of triarylmethane, a monoclonal antibody or a polyethylene glycol derivative.

The invention also provides a pharmaceutical composition comprising in a pharmaceutically-acceptable carrier an IMiD™ and at least one cytokine. In a specific embodiment, said cytokine is erythropoietin (Epo), SCF, GM-CSF, Flt-3L, TNFα, IL-3, or any combination thereof.

The invention further provides a method of treating an individual having a hemoglobinopathy or anemia, said method comprising administering to said individual a compound in an amount and for a time sufficient to cause a detectable increase in the level of alpha hemoglobin stabilizing protein (AHSP). In one embodiment of the method, said compound is an IMiD™. In a specific embodiment, said compound is α-(3-aminophthalimido) glutarimide (also known as 4-(Amino)-2-(2,6-dioxo(3-piperidyl))-isoindoline-1,3-dione) or 3-(4′aminoisoindoline-1′-one)-1-piperidine-2,6-dione.

As used herein, the term “hemoglobinopathy” means any defect in the structure or function of any hemoglobin of an individual, and includes defects in the primary, secondary, tertiary or quaternary structure of hemoglobin caused by any mutation, such as deletion mutations or substitution mutations in the coding regions of any hemoglobin gene, or mutations in, or deletions of, the promoters or enhancers of such genes that cause a reduction in the amount of hemoglobin produced as compared to a normal or standard condition. The term further includes any decrease in the amount or effectiveness of hemoglobin, whether normal or abnormal, caused by external factors such as disease, chemotherapy, toxins, poisons, or the like.

As used herein, “anemia” means any reduction in the amount of hemoglobin in the bloodstream as compared to the normal condition. Such reduction may be due to a loss of blood cells, a deficit of iron, toxins, poisons, disease, or any other physiological cause.

As used herein, the terms “symptom of a hemoglobinopathy” and “symptom of anemia” means any physiological or biological symptom associated with any hemoglobinopathy or anemia, including but not limited to dizziness, shortness of breath, loss of consciousness, tiredness, weakness, hemolysis, pains associated with abnormal hemoglobin, reduced erythrocyte counts (i.e., reduced hematocrit), a reduced ability of a given volume of blood to carry oxygen, as compared with a volume of normal blood, deformities of erythrocytes visible under a microscope, etc. The term also includes negative psychological symptoms such as depression, low self-esteem, perception of illness, perception of limited physical capability, etc.

As used herein, the term “IMiD” means that class of compounds disclosed in Section 5.2, below, including the compounds 4-(Amino)-2-(2,6-dioxo(3-piperidyl))-isoindoline-1,3-dione (also known as α-(3-aminophthalimido) glutarimide) and 3-(4′aminoisoindoline-1′-one)-1-piperidine-2,6-dione.

As used herein, the terms “CC-5013” and “Revimid™” mean the compound 3-(4-amino-1-oxo-1,3-dihydroisoindol-2-yl)-piperidine-2,6-dione (also known as 3-(4′aminoisolindoline-1′-onw)-1-piperidine-2,6-dione).

As used herein, the terms “CC-4047” and “Actimid™” mean the compound 4-(Amino)-2-(2,6-dioxo(3-piperidyl))-isoindoline-1,3-dione (also known as α-(3-aminophthalimido) glutarimide).

As used herein, the term “CD34⁺ cells” means CD34⁺ stem, progenitor, or precursor cells.

As used herein, the terms HEMOXIN™ and NIPRISAN™ refer to the plant extract as described in U.S. Pat. No. 5,800,819, characterized by a mixture of about 12 to about 17 parts by weight of Piper guineense seeds, from about 15 to about 19 parts by weight of Pterocarpus osun stem, from about 12 to about 18 parts by weight of Eugenia caryophyllata fruit, and from about 25 to about 32 parts by weight of Sorghum bicolor leaves, and optionally 15-22 parts by weight potash, wherein the mixture is extracted with cold water. This plant extract has antisickling activity.

### 4. DESCRIPTION OF THE FIGURES

FIG. 1 depicts the timeline of CD34⁺ cell differentiation in the presence of SCF, Flt3-L, GM-CSF and TNFα, either in the presence of DMSO (control) or 4-(Amino)-2-(2,6-dioxo(3-piperidyl))-isoindoline-1,3-dione.

FIG. 2 depicts the induction of expression of fetal hemoglobin genes hemoglobin εₗ, hemoglobin γ_{A} and hemoglobin γ_{B} in response to DMSO (control) or 4-(Amino)-2-(2,6-dioxo(3-piperidyl))-isoindoline-1,3-dione. Also depicted is the effect of 4-(Amino)-2-(2,6-dioxo(3-piperidyl))-isoindoline-1,3-dione (CC-4047) on the induction of ESTs related to hemoglobin εₗ.

FIG. 3 depicts the level of the marker glycophorin A in CD34⁺ cells in the presence of 0, 0.01, 0.1, 1.0, 10 or 100 μM 4-(Amino)-2-(2,6-dioxo(3-piperidyl))-isoindoline-1,3-dione or 3-(4-amino-1-oxo-1,3-dihydroisoindol-2-yl)-piperidine-2,6-dione after six days of culture.

FIG. 4 depicts the level of fetal hemoglobin in CD34⁺ cells in the presence of 0, 0.01, 0.1, 1.0, 10 or 100 μM 4-(Amino)-2-(2,6-dioxo(3-piperidyl))-isoindoline-1,3-dione or 3-(4-amino-1-oxo-1,3-dihydroisoindol-2-yl)-piperidine-2,6-dione after six days of culture

FIG. 5 depicts a portion of a microarray showing the relative expression levels of erythroid-specific genes at 0, 3 and 6 days of culture in medium containing SCF, Flt3-L, GM-CSF and TNFα. Expression levels were determined by hybridization of RNA-derived biotin-labeled cRNA to an Affymetrix U133A microarray.

FIG. 6 depicts the timeline of CD34⁺ cell expansion in the presence of SCF, Flt3-L and IL-3, followed by differentiation in the presence of SCF and erythropoietin, either in the presence of DMSO (control) or 4-(Amino)-2-(2,6-dioxo(3-piperidyl))-isoindoline-1,3-dione.

FIG. 7 depicts the results of a FACS analysis showing a slight decrease in glycophorin A expression after differentiation in the presence of Epo and SCF in the presence of 4-(Amino)-2-(2,6-dioxo(3-piperidyl))-isoindoline-1,3-dione or DMSO (control). Numbers in each quadrant indicate the percentage of cells expressing glycophorin A and/or CD71.

FIG. 8 depicts the increase in fetal hemoglobin expression in CD34⁺ cell differentiated for 6 days in the presence of 4-(Amino)-2-(2,6-dioxo(3-piperidyl))-isoindoline-1,3-dione as compared to a DMSO control, and SCF (50 ng/ml)+Epo (4 units/ml). 4-(Amino)-2-(2,6-dioxo(3-piperidyl))-isoindoline-1,3-dione concentrations were varied from 0.001 μM to 10 μM. Data points indicate the percentage of cells identified by flow cytometry expressing fetal hemoglobin.

FIG. 9 depicts a FACS analysis showing that the increase in fetal hemoglobin expression (Y-axis) is associated with a decrease in adult hemoglobin expression. Numbers in each quadrant indicate the percentage of cells expressing fetal hemoglobin and/or adult hemoglobin. Cells were differentiated for 6 days in the presence of Epo, SCF, and either 4-(Amino)-2-(2,6-dioxo(3-piperidyl))-isoindoline-1,3-dione or DMSO.

FIG. 10 depicts the increase in expression of fetal hemoglobin due to 4-(Amino)-2-(2,6-dioxo(3-piperidyl))-isoindoline-1,3-dione over that induced by hydroxyurea or 5-azacytidine. Cells were cultured for six days in the presence of SCF (50 ng/ml) and Epo (2 U/ml), and either DMSO (control), 4-(Amino)-2-(2,6-dioxo(3-piperidyl))-isoindoline-1,3-dione (0.1, 1, 10 μM), 5-azacytidine (0.1, 1 μM; toxic at 10 μM) or hydroxyurea (0.1, 1, 10 μM). Bars indicate the percentage of cells demonstrating fetal hemoglobin expression.

FIG. 11 depicts flow cytometry analysis showing a synergy between 4-(Amino)-2-(2,6-dioxo(3-piperidyl))-isoindoline-1,3-dione and hydroxyurea in increasing fetal hemoglobin expression. CD34⁺ cells were differentiated for six days in the presence of SCF and Epo, as above, and either 3-(4-amino-1-oxo-1,3-dihydroisoindol-2-yl)-piperidine-2,6-dione or 4-(Amino)-2-(2,6-dioxo(3-piperidyl))-isoindoline-1,3-dione (see Section 5.2). Numbers in each panel indicate the percentage of cells expressing fetal hemoglobin.

FIG. 12 depicts gels of STAT5 from UT-7 in the presence or absence of Epo, and with either 4-(Amino)-2-(2,6-dioxo(3-piperidyl))-isoindoline-1,3-dione or DMSO (control). Lower panel: absolute level of STAT5 protein. Top panel: level of phosphorylated STAT5 protein.

### 5. DETAILED DESCRIPTION OF THE INVENTION

5.1. Differentiation of CD34⁺ Cells to an Erythroid Lineage

The present invention provides methods of modulating the differentiation of CD34⁺ stem, precursor or progenitor cells to a predominantly erythroid lineage. The inventors have discovered that the class of immunomodulatory compounds known as IMiDs™, when contacted with such cells under the appropriate conditions, cause a shift in differentiation towards an erythroid lineage. This shift in differentiation is evidenced by hallmark changes in gene expression, including but not limited to increases in the expression of genes encoding glycophorin A, and fetal hemoglobins such as hemoglobin γ and hemoglobin ε. Thus, the method of the present invention is highly useful in that it provides a means for enhancing the production of a population of hemoglobin-producing cells that can substitute for the naturally-occurring population of hemoglobin-producing cells of an individual.

IMiDs™ also cause the increase in expression in differentiated CD34⁺ cells of alpha hemoglobin stabilizing protein, a protein that preferentially binds alpha hemoglobin, but not beta hemoglobin or hemoglobin A (Hbα₂β₂). This is advantageous because alpha hemoglobin in excess of beta hemoglobin tends to form precipitates that damage red blood cells. As such, AHSP, and an IMiD-mediated increase in AHSP expression, is predicted to modulate pathological states of alpha hemoglobin excess, including beta thalassemia. Such an effect on the expression of AHSP, coupled with enhancement of fetal hemoglobin expression, is an advantage of IMiD treatment versus other drugs that increase the expression of fetal hemoglobin.

Thus, the invention first provides a method of modulating the differentiation of a CD34⁺ cell to an erythroid lineage comprising differentiating said cell under suitable conditions and in the presence of an immunomodulatory compound such as an IMiD, or a pharmaceutically acceptable salt, solvate, hydrate, stereoisomer, clathrate or prodrug thereof. Examples of IMiDs™ that may be used in the present invention are described in detail in Section 5.2, below. However, particularly preferred IMiDs™ are 3-(4-amino-1-oxo-1,3-dihydroisoindol-2-yl)-piperidine-2,6-dione and 4-(Amino)-2-(2,6-dioxo(3-piperidyl))-isoindoline-1,3-dione.

The CD34⁺ cell may be any stem, progenitor, or committed cell able to differentiate into an erythroid cell. Such cells may be totipotent or pluripotent, or may be committed to a hematopoietic lineage. The CD34 + cell may be derived from any source; particularly preferred are “embryonic-like” stem cells derived from the placenta. For a description of such embryonic-like stem cells and methods of obtaining them, see U.S. application publication No. 2003/0180269 A1, published Sep. 25, 2003, which is incorporated by reference herein in its entirety. Other CD34⁺ cells useful for the methods of the invention include stem cells obtained from any tissue (such as, for example, hematopoietic stem cells or embryonic stem cells) and non-committed progenitor cells from any tissue. Such CD34 ⁺ cells may be heterologous or autologous with reference to the intended recipient, when such cells, the differentiation of which is modulated according to the methods of the present invention, are used to treat anemia or a hemoglobinopathy.

Differentiation of the CD34⁺ cells may typically take place over the course of 3-6 days. In in vitro assays in which CD34⁺ cells were cultured in the presence of an IMiD (described in the Examples), changes in gene expression indicating differentiation along an erythroid pathway were evident by the third day of culture. Erythroid-specific gene expression was significantly increased, and phenotypic characteristics of erythroid cells were present in the CD34⁺ cells by day 6 of culture.

According to the invention, therefore, CD34⁺ cells may be cultured in vitro in the presence of a compound of the invention, such as an immunomodulatory compound, specifically, an IMiD, for a period of days sufficient for erythroid-specific gene expression, particularly fetal hemoglobin gene expression, and/or cell characteristics to appear. In various embodiments, the CD34⁺ cells may be cultured for 3, 6, 9 or 12 days, or more. The compound of the invention may be introduced once at the start of culture, and culturing continued until differentiation is substantially complete, or for 3, 6, 9, 12 or more days. Alternatively, the compound of the invention may be administered to a culture of CD34⁺ cells a plurality of times during culture. The CD34⁺ cells may be cultured and differentiated in the presence of a single compound of the invention, or in the presence of a plurality of different compounds of the invention.

The compounds of the invention may be used at any concentration from 0.01 μM-10 mM. Preferably, the concentration for 4-(Amino)-2-(2,6-dioxo(3-piperidyl))-isoindoline-1,3-dione is between 0.01-10 μM, and for 3-(4-amino-1-oxo-1,3-dihydroisoindol-2-yl)-piperidine-2,6-dione the concentration is preferably 0.01-100 μM.

In addition to differentiating CD34⁺ cells in vitro, such cells may be differentiated within an individual, in vivo. Such an individual is preferably a mammal, even more preferably a human. As with in vitro differentiation of CD34⁺ cells, CD34⁺ cells within an individual may be differentiated by administration of one or more of the immunomodulatory compounds of the invention. Such administration may be in the form of a single dose. Alternatively, the individual may be administered the one or more compounds of the invention a plurality of times. Such administration may be performed, for example, over a period of 3, 6, 9, 12 or more days, and may follow the dosing regimen(s) and forms described in Section 5.4, below.

Where differentiation of CD34⁺ cells is to be accomplished in vivo, differentiation may be accomplished using the immunomodulatory compounds alone, or a combination of immunomodulatory compounds and one or more cytokines. For example, for an individual having a hemoglobinopathy such as sickle cell anemia or a thalassemia, who has a higher than normal level of SCF and/or erythropoietin, in vivo differentiation may be accomplished by administration of one or more of the immunomodulatory compounds (e.g., 4-(Amino)-2-(2,6-dioxo(3-piperidyl))-isoindoline-1,3-dione). Conversely, where an individual suffers an anemia that is the result of, or is characterized by, a lower-than-normal level of erythropoietic cytokines (e.g., SCF or erythropoietin), such cytokines may be administered along with, or prior to, administration of the immunomodulatory compound. For example, an individual suffering from chemotherapy-induced anemia may be administered one or more cytokines (e.g., the combination of SCF, Flt-3L and IL-3) for, e.g., 3-6 days, followed by administration for, e.g., 3-6 days, of one or more immunomodulatory compounds of the invention, particularly with SCF and erythropoietin, in an amount sufficient to cause a detectable increase in fetal hemoglobin expression in CD34⁺ cells of said individual. Alternatively, such individual may be administered CD34⁺ cells contacted with one or more cytokines in vitro (e.g., SCF, Flt-3L and IL-3) for, e.g., 3-6 days, followed by administration of the cells to the individual, along with SCF and erythropoietin in an amount sufficient to cause a detectable increase in fetal hemoglobin expression in the CD34⁺ cells. Such administration may be performed a single time or multiple times, and any one or more of such administrations may be accompanied by the administration of a compound of the invention (see Section 5.3), a second compound (see below), or a combination of all three.

Any of the compounds of the invention (e.g., 4-(Amino)-2-(2,6-dioxo(3-piperidyl))-isoindoline-1,3-dione or 3-(4-amino-1-oxo-1,3-dihydroisoindol-2-yl)-piperidine-2,6-dione) may be contacted with a CD34⁺ stem, progenitor or precursor cell in order to induce one or more genes in the cell that are associated with or necessary for erythropoiesis and/or hematopoiesis, in particular, one or more genes encoding a fetal hemoglobin. In one embodiment, the invention provides a method of inducing one or more genes associated with or essential for erythropoiesis or hematopoiesis, comprising contacting an hematopoietic stem, progenitor or precursor cell with an immunomodulatory agent in the presence of erythropoietin and stem cell factor, wherein said immunomodulatory agent is present in a sufficient amount to cause said hematopoietic stem, progenitor or precursor cell to express one or more genes encoding fetal hemoglobin. In a specific embodiment, said hematopoietic stem, progenitor or precursor cell is a CD34⁺ cell. In another specific embodiment, said one or more genes associated with or essential for erythropoiesis or hematopoiesis are genes encoding Kruppel-like factor 1 erythroid; rhesus blood group-associated glycoprotein; glycophorin B; integrin alpha 2b; erythroid-associated factor; glycophorin A; Kell blood group precursor; hemoglobin α2; solute carrier 4, anion exchanger; carbonic anhydrase 1 hemoglobin γA; hemoglobin γG; hemoglobin ε1; or any combination of the foregoing. In another specific embodiment, said immunomodulatory agent is an IMiD™. In a more specific embodiment, said IMiD™ is a-(3-aminophthalimido) glutarimide; an analog or prodrug of α-(3-aminophthalimido) glutarimide; 3-(4′aminoisoindoline-1′-one)-1-piperidine-2,6-dione; an analog or prodrug of 3-(4′aminoisoindoline-1′-one)-1-piperidine-2,6-dione; or a compound of the formula

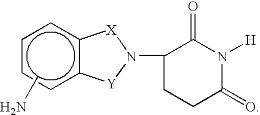

In addition to one or more compounds of the invention, the CD34⁺ cells may additionally be differentiated, either in vivo or in vitro, in the presence of one or more cytokines. Cytokines useful to direct CD34⁺ cells along an erythroid differentiation pathway include, but are not limited to, erythropoietin (Epo), TNFα, stem cell factor (SCF), Flt-3L, and granulocyte macrophage-colony stimulating factor (GM-CSF). Epo and SCF are known to be erythropoietic cytokines. Thus, in one embodiment, CD34⁺ cells are differentiated in the presence of Epo or SCF. In another, preferred, embodiment, the CD34⁺ cells are differentiated in the presence of Epo and SCF. In another embodiment, the CD34⁺ cells are differentiated in the presence of the combination of TNFα, SCF, Flt-3L and GM-CSF. In another embodiment, said cells that are differentiated are one or more cells in cell culture. In another embodiment, said cells that are differentiated are cells within an individual. In an embodiment of in vitro differentiation, one or more of Epo, TNFα, SCF, Flt-3L and GM-CSF is contacted with one or more IMiDs™. In an embodiment of in vivo differentiation, one or more of Epo, TNFα, SCF, Flt-3L and GM-CSF is administered to an individual in the same treatment regimen as the one or more IMiDs™.

The cytokines used in the methods of the invention may be naturally-occurring cytokines, or may be an artificial derivative or analog of the cytokines. For example, analogs or derivatives of erythropoietin that may be used in combination with the compounds of the invention include, but are not limited to, Aranesp™ and Darbopoietin™.

Cytokines used may be purified from natural sources or recombinantly produced. Examples of recombinant cytokines that may be used in the methods of the invention include filgrastim, or recombinant granulocyte-colony stimulating factor (G-CSF), which is sold in the United States under the trade name Neupogen™ (Amgen, Thousand Oaks, Calif.); sargramostim, or recombinant GM-CSF, which is sold in the United States under the trade name Leukine™ (Immunex, Seattle, Wash.); recombinant Epo, which is sold in the United States under the trade name Epogen™ (Amgen, Thousand Oaks, Calif.); and methionyl stem cell factor (SCF), which is sold in the United States under the trade name Ancestim. Recombinant and mutated forms of GM-CSF can be prepared as described in U.S. Pat. Nos. 5,391,485; 5,393,870; and 5,229,496; all of which are incorporated herein by reference. Recombinant and mutated forms of G-CSF can be prepared as described in U.S. Pat. Nos. 4,810,643; 4,999,291; 5,528,823; and 5,580,755; all of which are incorporated herein by reference.

Other cytokines may be used which encourage the survival and/or proliferation of hematopoietic precursor cells and immunologically active poietic cells in vitro or in vivo, or which stimulate the division and differentiation of committed erythroid progenitors in cells in vitro or in vivo. Such cytokines include, but are not limited to: interleukins, such as IL-2 (including recombinant IL-II (“rIL2”) and canarypox IL-2), IL-10, IL-12, and IL-18; interferons, such as interferon alfa-2a, interferon alfa-2b, interferon alfa-n1, interferon alfa-n3, interferon beta-I a, and interferon gamma-I b; and G-CSF.

When administered to a person having a hemoglobinopathy, the compounds of the invention, particularly in the presence of Epo, particularly in the presence of the combination of TNFα, SCF, Flt-3L and GM-CSF, and more particularly in the presence of Epo and SCF, induce the production of erythrocytes, and the production of fetal hemoglobin as well as the production of AHSP. As noted above, cytokines used may include purified or recombinant forms, or analogs or derivatives of specific cytokines.

The compounds of the invention may also be administered in conjunction with one or more second compounds known to have, or suspected of having, a beneficial effect on a hemoglobinopathy. In this context, “beneficial effect” means any reduction of any symptom of a hemoglobinopathy or anemia.

For example, with specific reference to the hemoglobinopathy sickle cell anemia, the second compound can be a compound, other than a compound of the invention, that is known or suspected to induce the production of fetal hemoglobin. Such compounds include hydroxyurea, and butyrates or butyrate derivatives. The second compound may also be a compound that relaxes blood vessels, such as nitrous oxide, e.g., exogenously-applied or administered nitrous oxide. The second compound may also be a compound that binds directly to hemoglobin S, preventing it from assuming the sickle-inducing conformation. For example, the plant extract known as HEMOXIN™ (NIPRISAN™; see U.S. Pat. No. 5,800,819), which is an extract of a mixture of about 12 to about 17 parts by weight of Piper guineense seeds, from about 15 to about 19 parts by weight of Pterocarpus osun stem, from about 12 to about 18 parts by weight of Eugenia caryophyllata fruit, and from about 25 to about 32 parts by weight of Sorghum bicolor leaves, and optionally 15-22 parts by weight potash, wherein the mixture is extracted with cold water, has antisickling activity. The second compound may also be a Gardos channel antagonist. Examples of Gardos channel antagonists include clotrimazole and triaryl methane derivatives. The second compound may also be one that reduces red blood cell adhesion, thereby reducing the amount of clotting pervasive in sickle cell anemia.

Other hemoglobinopathies may be treated with a second compound known or suspected to be efficacious for the specific condition. For example, β thalassemia may additionally be treated with the second compounds Deferoxamine, an iron chelator that helps prevent the buildup of iron in the blood, or folate (vitamin B9). Thalassemia or sickle cell anemia may also be treated with protein C as the second compound (U.S. Pat. No. 6,372,213). There is some evidence that herbal remedies can ameliorate symptoms of hemoglobinopathies, e.g., thalassemia; such remedies, and any of the specific active compounds contained therein, may also be used as a second compound in the method of the invention. See, e.g., Wu Zhikui et al. “The Effect of Bushen Shengxue Fang on β-thalassemia at the Gene Level,” Journal of Traditional Chinese Medicine 18(4): 300-303 (1998); U.S. Pat. No. 6,538,023 “Therapeutic Uses of Green Tea Polyphenols for Sickle Cell Disease”. Treatment of autoimmune hemolytic anemia can include corticosteroids as the second compound.

Second compounds that are proteins may also be derivatives or analogs of other proteins. Such derivatives may include, but are not limited to, proteins that lack carbohydrate moieties normally present in their naturally occurring forms (e.g., nonglycosylated forms), pegylated derivatives and fusion proteins, such as proteins formed by fusing IgG1 or IgG3 to the protein or active portion of the protein of interest. See, e.g., Penichet, M. L. and Morrison, S. L., J. Immunol. Methods 248:91-101 (2001).

Cytokines and/or other compounds potentially useful in the treatment of anemia or a hemoglobinopathy may be administered at the same time as the immunomodulatory compounds useful in the present invention. In this regard, the cytokines or other compounds may be administered as formulations separate from the immunomodulatory compounds, or, where possible, may be compounded with the immunomodulatory compounds for administration as a single pharmaceutical composition. Alternatively, the cytokines, the other compounds, or both, may be administered separately from the immunomodulatory compounds used in the methods of the invention, and may follow the same or different dosing schedules. In a preferred embodiment, the immunomodulatory compounds, e.g. IMiDs™, cytokines, and any other compound useful to treat anemia or a hemoglobinopathy, are administered at the same time, but in separate pharmaceutical formulations for flexibility in administration.

In addition to the treatment combinations outlined above, the treated individual may be given transfusions. Such transfusions may be of blood, preferably matched blood, or of a blood substitute such as Hemospan™ or Hemospan™ PS (Sangart).

In any of the treatment combinations described herein, the treated individual is eukaryotic. Preferably, the treated individual is a mammal, and even more preferably, human.

The methods of the invention may be used to treat any anemia, including anemia resulting from a hemoglobinopathy. Hemoglobinopathies and anemias treatable by the methods of the invention may be genetic in origin, such as sickle-cell anemia or thalassemias. The hemoglobinopathy may be due to a disease, such as cancer, including, but not limited to, cancers of the hematopoietic or lymphatic systems. Other conditions treatable using the methods of the invention include hypersplenism, splenectomy, bowel resection, and bone marrow infiltration. The methods of the present invention may also be used to treat anemia resulting from the deliberate or accidental introduction of a poison, toxin or drug. For example, anemias resulting from cancer chemotherapies may be treated using the methods and compounds of the invention. As such, the methods of the invention may be employed when anemia or a hemoglobinopathy is the primary condition to be treated, or is a secondary condition caused by an underlying disease or treatment regimen.

### 5.2. THE COMPOUNDS OF THE INVENTION

Compounds of the invention can either be commercially purchased or prepared according to the methods described in the patents or patent publications disclosed herein. Further, optically pure compositions can be asymmetrically synthesized or resolved using known resolving agents or chiral columns as well as other standard synthetic organic chemistry techniques. Compounds used in the invention may include immunomodulatory compounds that are racemic, stereomerically enriched or stereomerically pure, and pharmaceutically acceptable salts, solvates, stereoisomers, and prodrugs thereof.

Preferred compounds used in the invention are small organic molecules having a molecular weight less than about 1,000 g/mol, and are not proteins, peptides, oligonucleotides, oligosaccharides or other macromolecules.

As used herein and unless otherwise indicated, the terms “immunomodulatory compounds” and “IMiDs™” (Celgene Corporation) encompasses small organic molecules that markedly inhibit TNF-a, LPS induced monocyte IL 1β and IL12, and partially inhibit IL6 production. Specific immunomodulatory compounds are discussed below.

TNF-α is an inflammatory cytokine produced by macrophages and monocytes during acute inflammation. TNF-α is responsible for a diverse range of signaling events within cells. Without being limited by theory, one of the biological effects exerted by the immunomodulatory compounds of the invention is the reduction of synthesis of TNF-α. Immunomodulatory compounds of the invention enhance the degradation of TNF-α mRNA.

Further, without being limited by theory, immunomodulatory compounds used in the invention may also be potent co-stimulators of T cells and increase cell proliferation dramatically in a dose dependent manner. Immunomodulatory compounds of the invention may also have a greater co-stimulatory effect on the CD8+ T cell subset than on the CD4+ T cell subset. In addition, the compounds preferably have anti-inflammatory properties, and efficiently co-stimulate T cells. Further, without being limited by a particular theory, immunomodulatory compounds used in the invention may be capable of acting both indirectly through cytokine activation and directly on Natural Killer (“NK”) cells, and increase the NK cells' ability to produce beneficial cytokines such as, but not limited to, IFN-γ.

Specific examples of immunomodulatory compounds, include, but are not limited to, cyano and carboxy derivatives of substituted styrenes such as those disclosed in U.S. Pat. No. 5,929,117; 1-oxo-2-(2,6-dioxo-3-fluoropiperidin-3yl) isoindolines and 1,3-dioxo-2-(2,6-dioxo-3-fluoropiperidine-3-yl) isoindolines such as those described in U.S. Pat. Nos. 5,874,448 and 5,955,476; the tetra substituted 2-(2,6-dioxopiperdin-3-yl)-1-oxoisoindolines described in U.S. Pat. No. 5,798,368; 1-oxo and 1,3-dioxo-2-(2,6-dioxopiperidin-3-yl) isoindolines (e.g., 4-methyl derivatives of thalidomide), including, but not limited to, those disclosed in U.S. Pat. Nos. 5,635,517, 6,476,052, 6,555,554, and 6,403,613; 1-oxo and 1,3-dioxoisoindolines substituted in the 4- or 5-position of the indoline ring (e.g., 4-(4-amino-1,3-dioxoisoindoline-2-yl)-4-carbamoylbutanoic acid) described in U.S. Pat. No. 6,380,239; isoindoline-1-one and isoindoline-1,3-dione substituted in the 2-position with 2,6-dioxo-3-hydroxypiperidin-5-yl (e.g., 2-(2,6-dioxo-3-hydroxy-5-fluoropiperidin-5-yl)-4-aminoisoindolin-1 -one) described in U.S. Pat. No. 6,458,810; a class of non-polypeptide cyclic amides disclosed in U.S. Pat. Nos. 5,698,579 and 5,877,200; aminothalidomide, as well as analogs, hydrolysis products, metabolites, derivatives and precursors of aminothalidomide, and substituted 2-(2,6-dioxopiperidin-3-yl) phthalimides and substituted 2-(2,6-dioxopiperidin-3-yl)-1-oxoisoindoles such as those described in U.S. Pat. Nos. 6,281,230 and 6,316,471; and isoindole-imide compounds such as those described in U.S. patent application no. 09/972,487 filed on Oct. 5, 2001, U.S. patent application no. 10/032,286 filed on Dec. 21, 2001, and International Application No. PCT/US01/50401 (International Publication No. WO 02/059106). The entireties of each of the patents and patent applications identified herein are incorporated herein by reference. Immunomodulatory compounds do not include thalidomide.

Other specific immunomodulatory compounds of the invention include, but are not limited to, 1-oxo-and 1,3dioxo-2-(2,6-dioxopiperidin-3-yl) isoindolines substituted with amino in the benzo ring as described in U.S. Pat. No. 5,635,517 which is incorporated herein by reference. These compounds have the structure I: 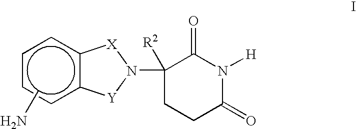
in which one of X and Y is C═O, the other of X and Y is C═O or CH₂, and R² is hydrogen or lower alkyl, in particular methyl. Specific immunomodulatory compounds include, but are not limited to:

1-oxo-2-(2,6-dioxopiperidin-3-yl)-4-aminoisoindoline;

1-oxo-2-(2,6-dioxopiperidin-3-yl)-5-aminoisoindoline;

1-oxo-2-(2,6-dioxopiperidin-3-yl)-6-aminoisoindoline;

1-oxo-2-(2,6-dioxopiperidin-3-yl)-7-aminoisoindoline;

1,3-dioxo-2-(2,6-dioxopiperidin-3-yl)-4-aminoisoindoline; and

1,3-dioxo-2-(2,6-dioxopiperidin-3-yl)-5-aminoisoindoline.

Other specific immunomodulatory compounds of the invention belong to a class of substituted 2-(2,6-dioxopiperidin-3-yl) phthalimides and substituted 2-(2,6-dioxopiperidin-3-yl)-1-oxoisoindoles, such as those described in U.S. Pat. Nos. 6,281,230; 6,316,471; 6,335,349; and 6,476,052, and International Patent Application No. PCT/US97/13375 (International Publication No. WO 98/03502), each of which is incorporated herein by reference. Representative compounds are of formula: 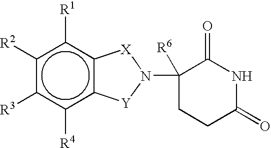
in which:

one of X and Y is C═O and the other of X and Y is C═O or CH₂;

(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen or alkyl of 1 to 8 carbon atoms;

R⁶ is hydrogen, alkyl of 1 to 8 carbon atoms, benzyl, or halo;

provided that R⁶ is other than hydrogen if X and Y are C═O and (i) each of R¹, R²,

R³, and R⁴ is fluoro or (ii) one of R¹, R², R³, or R⁴ is amino.

Compounds representative of this class are of the formulas:

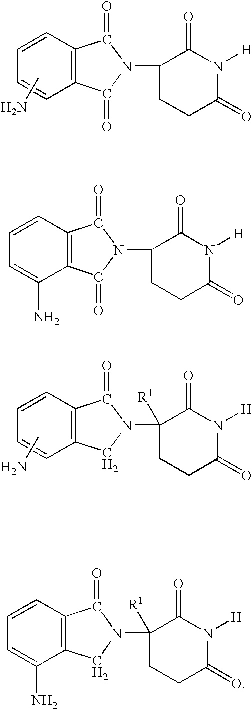

wherein R¹ is hydrogen or methyl. In a separate embodiment, the invention encompasses the use of enantiomerically pure forms (e.g. optically pure (R) or (S) enantiomers) of these compounds.

Still other specific immunomodulatory compounds of the invention belong to a class of isoindole-imides disclosed in U.S. patent application Publication Nos. U.S. 2003/0096841 and U.S. 2003/0045552, and International Application No. PCT/US01/50401 (International Publication No. WO 02/059106), each of which are incorporated herein by reference. Representative compounds are of formula II:

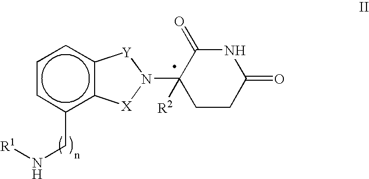

and pharmaceutically acceptable salts, hydrates, solvates, clathrates, enantiomers, diastereomers, racemates, and mixtures of stereoisomers thereof, wherein:

one of X and Y is C═O and the other is CH2 or C═O;

R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C_{1-C}₆)heterocycloalkyl, (C₀-C₄)alkyl-(C₂-C₅)heteroaryl, C(O)R₃, C(S)R₃, C(O)OR⁴, (C_{1-C}₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;

R² H, F benzyl, (C₁-C₈)alkyl, (C₂-C₈)alkenyl, or (C₂-C₈)alkynyl;

R³ and R³′ are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;

R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;

R⁵ is (C₁-C₈)alkyl, (C₂-C₈)alkenyl, (C₂-C₈)alkynyl, benzyl, aryl, or (C₂-C₅)heteroaryl;

each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O-R⁵ or the R⁶ groups can join to form a heterocycloalkyl group;

n is 0 or 1; and

* represents a chiral-carbon center.

In specific compounds of formula II, when n is 0 then R¹ is (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(O)OR⁴, (C₁-C₈)alkyl-N(R⁶)₂, (C₁-C₈)alkyl-OR⁵, (C₁-C₈)alkyl-C(O)OR⁵, C(S)NHR³, or (C₁-C₈)alkyl-O(CO)R⁵;

R² is H or (C₁-C₈)alkyl; and

R³ is (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₅-C₈)alkyl-N(R⁶)₂; (C₀-C₈)alkyl-NH—C(O)O-R⁵; (C₁-C₈)alkyl-OR⁵, (C₁-C₈)alkyl-(O)OR⁵, (C₁-C₈)alkyl-O(CO)R⁵, or C(O)OR⁵; and the other variables have the same definitions.

In other specific compounds of formula II, R² is H or (C₁-C₄)alkyl.

In other specific compounds of formula II, R¹ is (C₁-C₈)alkyl or benzyl.

In other specific compounds of formula II, R¹ is H, (C₁-C₈)alkyl, benzyl, CH₂OCH₃, CH₂CH₂OCH₃, or

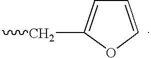

In another embodiment of the compounds of formula II, R¹ is

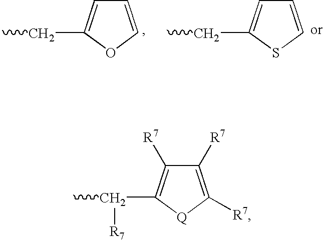

wherein Q is O or S, and each occurrence of R⁷ is independently H,(C₁-C₈)alkyl, (C₃-C₇)cycloalkyl, (C₂-C₈)alkenyl, (C₂-C₈)alkynyl, benzyl, aryl, halogen, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵, or adjacent occurrences of R⁷ can be taken together to form a bicyclic alkyl or aryl ring.

In other specific compounds of formula II, R¹ is C(O)R³.

In other specific compounds of formula II, R³ is (C₀-C₄)alkyl-(C₂-C₅)heteroaryl, (C₁-C₈)alkyl, aryl, or (C₀-C₄)alkyl-OR⁵.

In other specific compounds of formula II, heteroaryl is pyridyl, furyl, or thienyl.

In other specific compounds of formula II, R¹ is C(O)OR⁴.

In other specific compounds of formula II, the H of C(O)NHC(O) can be replaced with (C₁-C₄)alkyl, aryl, or benzyl.

Further examples of the compounds in this class include, but are not limited to: [2-(2,6-dioxo-piperidin-3-yl)-1,3-dioxo-2,3-dihydro-1H-isoindol-4-ylmethyl]-amide; (2-(2,6-dioxo-piperidin-3-yl)-1,3-dioxo-2,3-dihydro-1H-isoindol-4-ylmethyl)-carbamic acid tert-butyl ester; 4-(aminomethyl)-2-(2,6-dioxo(3-piperidyl))-isoindoline-1,3-dione; N-(2-(2,6-dioxo-piperidin-3-yl)-1,3-dioxo-2,3-dihydro-1H-isoindol-4-ylmethyl)-acetamide; N-{(2-(2,6-dioxo(3-piperidyl)-1,3-dioxoisoindolin-4-yl)methyl}cyclopropyl-carboxamide; 2-chloro-N-{(2-(2,6-dioxo(3-piperidyl))- 1,3-dioxoisoindolin-4-yl)methyl} acetamide; N-(2-(2,6-dioxo(3-piperidyl))-1,3-dioxoisoindolin-4-yl)-3-pyridylcarboxamide; 3-{1-oxo-4-(benzylamino)isoindolin-2-yl}piperidine-2,6-dione; 2-(2,6-dioxo(3-piperidyl))-4-(benzylamino)isoindoline-1,3-dione; N-{(2-(2,6-dioxo(3-piperidyl))-1,3-dioxoisoindolin-4-yl)methyl} propanamide; N-{(2-(2,6-dioxo(3-piperidyl))-1,3-dioxoisoindolin-4-yl)methyl}-3-pyridylcarboxamide; N-{(2-(2,6-dioxo(3-piperidyl))-1,3-dioxoisoindolin-4-yl)methyl} heptanamide; N-{(2-(2,6-dioxo(3-piperidyl))-1,3-dioxoisoindolin-4-yl)methyl}-2-furylcarboxamide; {N-(2-(2,6-dioxo(3-piperidyl))-1,3-dioxoisoindolin-4-yl)carbamoyl} methyl acetate; N-(2-(2,6-dioxo(3-piperidyl))-1,3-dioxoisoindolin-4-yl)pentanamide; N-(2-(2,6-dioxo(3-piperidyl))-1,3-dioxoisoindolin-4-yl)-2-thienylcarboxamide; N-{[2-(2,6-dioxo(3-piperidyl))-1,3-dioxoisoindolin-4-yl]methyl}(butylamino)carboxamide; N-{[2-(2,6-dioxo(3-piperidyl))-1,3-dioxoisoindolin-4-yl]methyl}(octylamino)carboxamide; and N-{[2-(2,6-dioxo(3-piperidyl))- 1,3-dioxoisoindolin-4-yl]methyl}(benzylamino)carboxamide.

Still other specific immunomodulatory compounds of the invention belong to a class of isoindole-imides disclosed in U.S. Patent Application Publication Nos. U.S. 2002/0045643, International Publication No. WO 98/54170, and U.S. Pat. No. 6,395,754, each of which is incorporated herein by reference. Representative compounds are of formula III: 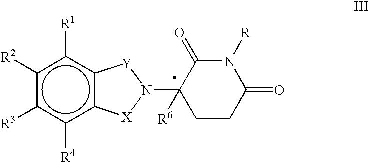
and pharmaceutically acceptable salts, hydrates, solvates, clathrates, enantiomers, diastereomers, racemates, and mixtures of stereoisomers thereof, wherein:

one of X and Y is C═O and the other is CH₂ or C═O;

R is H or CH₂OCOR′;

(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR⁵ and the remaining of R¹, R², R³, or R⁴ are hydrogen;

R⁵ is hydrogen or alkyl of 1 to 8 carbons

R⁶ hydrogen, alkyl of 1 to 8 carbon atoms, benzo, chloro, or fluoro;

R′ is R⁷-CHR¹⁰—N(R⁸R⁹);

R⁷ is m-phenylene or p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X₁CH₂CH₂— in.which X₁ is —O—, —S—, —NH—;

R¹⁰ is hydrogen, alkyl of to 8 carbon atoms, or phenyl; and

* represents a chiral-carbon center.

Other representative compounds are of formula:

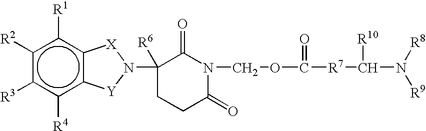

wherein:

one of X and Y is C═O and the other of X and Y is C═O or CH₂;

(i) each of R¹, R², R³, or R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen or alkyl of 1 to 8 carbon atoms;

R⁶ is hydrogen, alkyl of 1 to 8 carbon atoms, benzo, chloro, or fluoro;

R⁷ is m-phenylene or p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentarnethylene, hexamethylene, or —CH₂CH₂X¹CH₂CH₂— in which X¹ is —O—, —S—, —NH—;

R¹⁰ is hydrogen, alkyl of to 8 carbon atoms, or phenyl.

Other representative compounds are of formula:

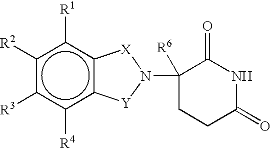

in which

one of X and Y is C═O and the other of X and Y is C═O or CH₂;

each of R¹, R², R³, and R⁴, independently of the others, is halo, alkyl of 1 to 4 carbon atoms, or alkoxy of 1 to 4 carbon atoms or (ii) one of R¹, R², R³, and R⁴ is nitro or protected amino and the remaining of R¹, R², R³, and R⁴ are hydrogen; and

R⁶ is hydrogen, alkyl of 1 to 8 carbon atoms, benzo, chloro, or fluoro.

Other representative compounds are of formula:

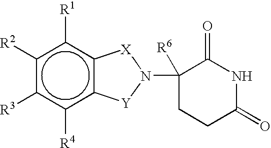

in which:

one of X and Y is C═O and the other of X and Y is C═O or CH₂;

(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen, alkyl of 1 to 8 carbon atoms, or CO—R⁷—CH(R¹⁰)NR⁸R⁹ in which each of R⁷, R⁸, R⁹, and R¹⁰ is as herein defined; and

R⁶ is alkyl of 1 to 8 carbon atoms, benzo, chloro, or fluoro.

Specific examples of the compounds are of formula:

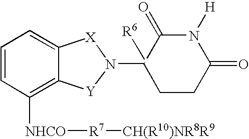

in which:

one of X and Y is C═O and the other of X and Y is C═O or CH₂;

R⁶ is hydrogen, alkyl of 1 to 8 carbon atoms, benzyl, chloro, or fluoro;

R⁷ is m-phenylene, p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is -O-, -S- or -NH-; and

R¹⁰ is hydrogen, alkyl of 1 to 8 carbon atoms, or phenyl.

Preferred immunomodulatory compounds of the invention are 4-(amino)-2-(2,6-dioxo (3-piperidyl))-isoindoline-1,3-dione and 3-(4-amino-1-oxo-1,3-dihydro-isoindol-2-yl)-piperidine-2,6-dione. The compounds can be obtained via standard, synthetic methods (see e.g., U.S. Pat. No. 5,635,517, incorporated herein by reference). The compounds are available from Celgene Corporation, Warren, N.J. 4-(Amino)-2-(2,6-dioxo(3-piperidyl))-isoindoline-1,3-dione has the following chemical structure: 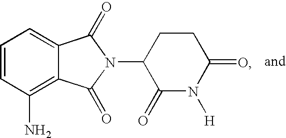
the compound 3-(4-amino-1-oxo-1,3-dihydro-isoindol-2-yl)-piperidine-2,6-dione has the following chemical structure:

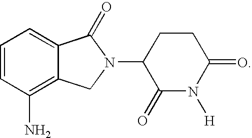

In another embodiment, specific immunomodulatory compounds of the invention encompass polymorphic forms of 3-(4-amino-1-oxo-1,3 dihydro-isoindol-2-yl)-piperidene-2,6-dione such as Form A, B, C, D, E, F, G and H, disclosed in U.S. provisional application No. 60/499,723 filed on Sep. 4, 2003, and the corresponding U.S. non-provisional application, filed Sep. 3, 2004, both of which are incorporated herein by reference. For example, Form A of 3-(4-amino-1-oxo-1,3 dihydro-isoindol-2-yl)-piperidene-2,6-dione is an unsolvated, crystalline material that can be obtained from non-aqueous solvent systems. Form A has an X-ray powder diffraction pattern comprising significant peaks at approximately 8, 14.5, 16, 17.5, 20.5, 24 and 26 degrees 2θ, and has a differential scanning calorimetry melting temperature maximum of about 270° C. Form A is weakly or not hygroscopic and appears to be the most thermodynamically stable anhydrous polymorph of 3-(4-amino-1-oxo-1,3 dihydro-isoindol-2-yl)-piperidine-2,6-dione discovered thus far.

Form B of 3-(4-amino-1-oxo-1,3 dihydro-isoindol-2-yl)-piperidene-2,6-dione is a hemihydrated, crystalline material that can be obtained from various solvent systems, including, but not limited to, hexane, toluene, and water. Form B has an X-ray powder diffraction pattern comprising significant peaks at approximately 16, 18, 22 and 27 degrees 20, and has endotherms from DSC curve of about 146 and 268° C., which are identified dehydration and melting by hot stage microscopy experiments. Interconversion studies show that Form B converts to Form E in aqueous solvent systems, and converts to other forms in acetone and other anhydrous systems.

Form C of 3-(4-amino-1-oxo-1,3 dihydro-isoindol-2-yl)-piperidene-2,6-dione is a hemisolvated crystalline material that can be obtained from solvents such as, but not limited to, acetone. Form C has an X-ray powder diffraction pattern comprising significant peaks at approximately 15.5 and 25 degrees 2θ, and has a differential scanning calorimetry melting temperature maximum of about 269° C. Form C is not hygroscopic below about 85% RH, but can convert to Form B at higher relative humidities.

Form D of 3-(4-amino-1-oxo-1,3 dihydro-isoindol-2-yl)-piperidene-2,6-dione is a crystalline, solvated polymorph prepared from a mixture of acetonitrile and water. Form D has an X-ray powder diffraction pattern comprising significant peaks at approximately 27 and 28 degrees 2θ, and has a differential scanning calorimetry melting temperature maximum of about 270° C. Form D is either weakly or not hygroscopic, but will typically convert to Form B when stressed at higher relative humidities.

Form E of 3-(4-amino-1-oxo-1,3 dihydro-isoindol-2-yl)-piperidene-2,6-dione is a dihydrated, crystalline material that can be obtained by slurrying 3-(4-amino-1-oxo-1,3 dihydro-isoindol-2-yl)-piperidene-2,6-dione in water and by a slow evaporation of 3-(4-amino-1-oxo-1,3 dihydro-isoindol-2-yl)-piperidene-2,6-dione in a solvent system with a ratio of about 9:1 acetone:water. Form E has an X-ray powder diffraction pattern comprising significant peaks at approximately 20, 24.5 and 29 degrees 2θ, and has a differential scanning calorimetry melting temperature maximum of about 269° C. Form E can convert to Form C in an acetone solvent system and to Form G in a THF solvent system. In aqueous solvent systems, Form E appears to be the most stable form. Desolvation experiments performed on Form E show that upon heating at about 125° C. for about five minutes, Form E can convert to Form B. Upon heating at 175° C. for about five minutes, Form B can convert to Form F.

Form F of 3-(4-amino-1-oxo-1,3 dihydro-isoindol-2-yl)-piperidene-2,6-dione is an unsolvated, crystalline material that can be obtained from the dehydration of Form E. Form F has an X-ray powder diffraction pattern comprising significant peaks at approximately 19, 19.5 and 25 degrees 2θ, and has a differential scanning calorimetry melting temperature maximum of about 269° C.

Form G of 3-(4-amino-1-oxo-1,3 dihydro-isoindol-2-yl)-piperidene-2,6-dione is an unsolvated, crystalline material that can be obtained from slurrying forms B and E in a solvent such as, but not limited to, tetrahydrofuran (THF). Form G has an X-ray powder diffraction pattern comprising significant peaks at approximately 21, 23 and 24.5 degrees 2θ, and has a differential scanning calorimetry melting temperature maximum of about 267° C.

Form H of 3-(4-amino-1-oxo-1,3 dihydro-isoindol-2-yl)-piperidene-2,6-dione is a partially hydrated (about 0.25 moles) crystalline material that can be obtained by exposing Form E to 0% relative humidity. Form H has an X-ray powder diffraction pattern comprising significant peaks at approximately 15, 26 and 31 degrees 20, and has a differential scanning calorimetry melting temperature maximum of about 269° C.

Other specific immunomodulatory compounds of the invention include, but are not limited to, 1-oxo-2-(2,6-dioxo-3-fluoropiperidin-3yl) isoindolines and 1,3-dioxo-2-(2,6-dioxo-3-fluoropiperidine-3-yl) isoindolines such as those described in U.S. Pat. Nos. 5,874,448 and 5,955,476, each of which is incorporated herein by reference. Representative compounds are of formula:

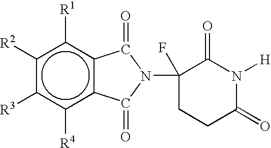

wherein Y is oxygen or H² and

each of R¹, R², R³, and R⁴, independently of the others, is hydrogen, halo, alkyl of 1 to 4 carbon atoms, alkoxy of 1 to 4 carbon atoms, or amino.

Other specific immunomodulatory compounds of the invention include, but are not limited to, the tetra substituted 2-(2,6-dioxopiperdin-3-yl)-1-oxoisoindolines described in U.S. Pat. No. 5,798,368, which is incorporated herein by reference. Representative compounds are of formula:

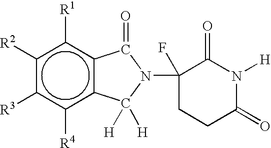

wherein each of R¹, R², R³, and R⁴, independently of the others, is halo, alkyl of 1 to 4 carbon atoms, or alkoxy of 1 to 4 carbon atoms.

Other specific immunomodulatory compounds of the invention include, but are not limited to, 1-oxo and 1,3-dioxo-2-(2,6-dioxopiperidin-3-yl) isoindolines disclosed in U.S. Pat. No. 6,403,613, which is incorporated herein by reference. Representative compounds are of formula:

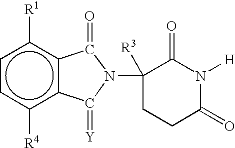

in which

Y is oxygen or H₂,

a first of R¹ and R² is halo, alkyl, alkoxy, alkylamino, dialkylamino, cyano, or carbamoyl, the second of R¹ and R², independently of the first, is hydrogen, halo, alkyl, alkoxy, alkylamino, dialkylamino, cyano, or carbamoyl, and

R³ is hydrogen, alkyl, or benzyl.

Specific examples of the compounds are of formula:

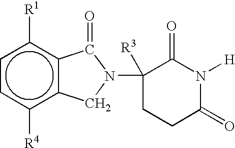

wherein a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl,

the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl, and

R³ is hydrogen, alkyl of from 1 to 4 carbon atoms, or benzyl. Specific examples include, but are not limited to, 1-oxo-2-(2,6-dioxopiperidin-3-yl)-4-methylisoindoline.

Other representative compounds are of formula:

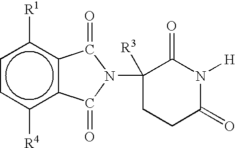

wherein a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl,

the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl, and

R³ is hydrogen, alkyl of from 1 to 4 carbon atoms, or benzyl.

Specific examples include, but are not limited to, 1-oxo-2-(2,6-dioxopiperidin-3-yl)-4-methylisoindoline.

Other specific immunomodulatory compounds of the invention include, but are not limited to, 1-oxo and 1,3-dioxoisoindolines substituted in the 4- or 5-position of the indoline ring described in U.S. Pat. No. 6,380,239 and co-pending U.S. application Ser No. 10/900,270, filed Jul. 28, 2004, which are incorporated herein by reference. Representative compounds are of formula:

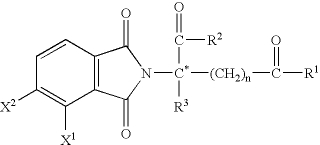

in which the carbon atom designated C* constitutes a center of chirality (when n is not zero and R¹ is not the same as R²); one of X¹ and X² is amino, nitro, alkyl of one to six carbons, or NH-Z, and the other of X¹ or X² is hydrogen; each of R¹ and R² independent of the other, is hydroxy or NH-Z; R³ is hydrogen, alkyl of one to six carbons, halo, or haloalkyl; Z is hydrogen, aryl, alkyl of one to six carbons, formyl, or acyl of one to six carbons; and n has a value of 0, 1, or 2; provided that if X¹ is amino, and n is 1 or 2, then R¹ and R² are not both hydroxy; and the salts thereof.

Further representative compounds are of formula:

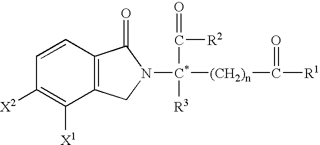

in which the carbon atom designated C* constitutes a center of chirality when n is not zero and R¹ is not R²; one of X¹ and X² is amino, nitro, alkyl of one to six carbons, or NH-Z, and the other of X¹ or X² is hydrogen; each of R¹ and R² independent of the other, is hydroxy or NH-Z; R³ is alkyl of one to six carbons, halo, or hydrogen; Z is hydrogen, aryl or an alkyl or acyl of one to six carbons; and n has a value of 0, 1, or 2.

Specific examples include, but are not limited to, 2-(4-amino-1-oxo-1,3-dihydro-isoindol-2-yl)-4-carbamoyl-butyric acid and 4-(4-amino-1-oxo-1,3-dihydro-isoindol-2-yl)-4-cabamoyl-butyric acid, which have the following structures, respectively, and pharmaceutically acceptable salts, solvates, prodrugs, and stereoisomers thereof:

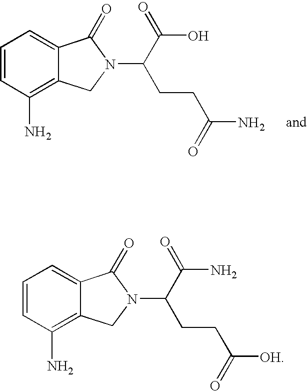

Other representative compounds are of formula: 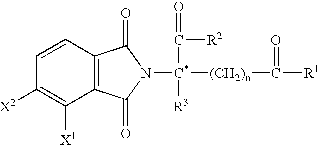
in which the carbon atom designated C* constitutes a center of chirality when n is not zero and R¹ is not R²; one of X¹ and X² is amino, nitro, alkyl of one to six carbons, or NH-Z, and the other of X¹ or X² is hydrogen; each of R¹ and R² independent of the other, is hydroxy or NH-Z; R³ is alkyl of one to six carbons, halo, or hydrogen; Z is hydrogen, aryl, or an alkyl or acyl of one to six carbons; and n has a value of 0, 1, or 2; and the salts thereof.

Specific examples include, but are not limited to, 4-carbamoyl-4-{4-[(furan-2-yl-methyl)-amino]-1,3-dioxo-1,3-dihydro-isoindol-2-yl}-butyric acid, 4-carbamoyl-2-{4-[(furan-2-yl-methyl)-amino]-1,3-dioxo- 1,3-dihydro-isoindol-2-yl }-butyric acid, 2-{4-[(furan-2-yl-methyl)-amino]-1,3-dioxo-1,3-dihydro-isoindol-2-yl}-4-phenylcarbamoyl-butyric acid, and 2-{4-[(furan-2-yl-methyl)-amino]-1,3-dioxo-1,3-dihydro-isoindol-2-yl}-pentanedioic acid, which have the following structures, respectively, and pharmaceutically acceptable salts, solvate, prodrugs, and stereoisomers thereof:

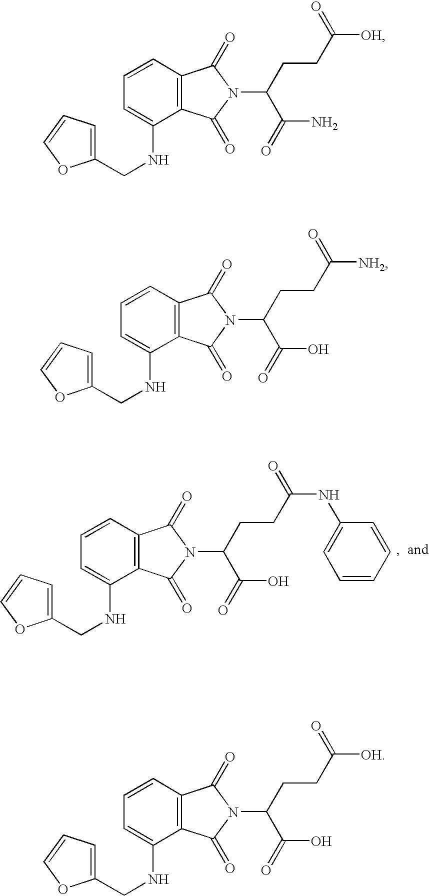

Other specific examples of the compounds are of formula:

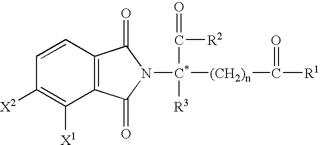

wherein one of X¹ and X² is nitro, or NH-Z, and the other of X¹ or X² is hydrogen;

each of R¹ and R², independent of the other, is hydroxy or NH-Z;

R³ is alkyl of one to six carbons, halo, or hydrogen;

Z is hydrogen, phenyl, an acyl of one to six carbons, or an alkyl of one to six carbons; and

n has a value of 0, 1, or 2;

provided that if one of X¹ and X² is nitro, and n is 1 or 2, then R¹ and R² are other than hydroxy; and

if —COR² and —(CH₂)ₙ,COR¹ are different, the carbon atom designated C* constitutes a center of chirality. Other representative compounds are of formula:

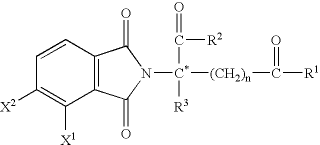

wherein one of X¹ and X² is alkyl of one to six carbons;

each of R¹ and R², independent of the other, is hydroxy or NH-Z;

R³ is alkyl of one to six carbons, halo, or hydrogen;

Z is hydrogen, phenyl, an acyl of one to six carbons, or an alkyl of one to six carbons; and

n has a value of 0, 1, or 2; and

if —COR² and —(CH₂)ₙCOR¹ are different, the carbon atom designated C* constitutes a center of chirality.

Still other specific immunomodulatory compounds of the invention include, but are not limited to, isoindoline-1-one and isoindoline-1,3-dione substituted in the 2-position with 2,6-dioxo-3-hydroxypiperidin-5-yl described in U.S. Pat. No. 6,458,810, which is incorporated herein by reference. Representative compounds are of formula:

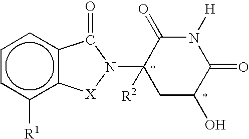

wherein:

the carbon atoms designated * constitute centers of chirality;

X is —C(O)— or —CH₂—;

R¹ is alkyl of 1 to 8 carbon atoms or —NHR³;

R² is hydrogen, alkyl of 1 to 8 carbon atoms, or halogen; and

R³ is hydrogen,

alkyl of 1 to 8 carbon atoms, unsubstituted or substituted with alkoxy of 1 to 8 carbon atoms, halo, amino, or alkylamino of 1 to 4 carbon atoms,

cycloalkyl of 3 to 18 carbon atoms,

phenyl, unsubstituted or substituted with alkyl of 1 to 8 carbon atoms, alkoxy of 1 to 8 carbon atoms, halo, amino, or alkylamino of 1 to 4 carbon atoms,

benzyl, unsubstituted or substituted with alkyl of 1 to 8 carbon atoms, alkoxy of 1 to 8 carbon atoms, halo, amino, or alkylamino of 1 to 4 carbon atoms, or —COR⁴ in which

R⁴ is hydrogen,

alkyl of 1 to 8 carbon atoms, unsubstituted or substituted with alkoxy of 1 to 8 carbon atoms, halo, amino, or alkylamino of 1 to 4 carbon atoms,

cycloalkyl of 3 to 18 carbon atoms,

phenyl, unsubstituted or substituted with alkyl of 1 to 8 carbon atoms, alkoxy of 1 to 8 carbon atoms, halo, amino, or alkylamino of 1 to 4 carbon atoms, or

benzyl, unsubstituted or substituted with alkyl of 1 to 8 carbon atoms, alkoxy of 1 to 8 carbon atoms, halo, amino, or alkylamino of 1 to 4 carbon atoms.

Compounds of the invention can either be commercially purchased or prepared according to the methods described in the patents or patent publications disclosed herein. Further, optically pure compounds can be asymmetrically synthesized or resolved using known resolving agents or chiral columns as well as other standard synthetic organic chemistry techniques.

As used herein and unless otherwise indicated, the term “pharmaceutically acceptable salt” encompasses non-toxic acid and base addition salts of the compound to which the term refers. Acceptable non-toxic acid addition salts include those derived from organic and inorganic acids or bases know in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

As used herein, and unless otherwise specified, the term “solvate” means a compound of the present invention or a salt thereof, that further includes a stoichiometric or non-stoichiometric amount of solvent bound by non-covalent intermolecular forces. Where the solvent is water, the solvate is a hydrate.

As used herein and unless otherwise indicated, the term “prodrug” means a derivative of a compound that can hydrolyze, oxidize, or otherwise react under biological conditions (in vitro or in vivo) to provide the compound. Examples of prodrugs include, but are not limited to, derivatives of immunomodulatory compounds of the invention that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of immunomodulatory compounds of the invention that comprise —NO, —NO₂, —ONO, or —ONO₂ moieties. Prodrugs can typically be prepared using well-known methods, such as those described in 1 Burger's Medicinal Chemistry and Drug Discovery, 172-178, 949-982 (Manfred E. Wolff ed., 5th ed. 1995), and Design of Prodrugs (H. Bundgaard ed., Elselvier, New York 1985).

As used herein and unless otherwise indicated, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxymethyl, pivaloyloxymethyl, and pivaloyloxyethyl esters), lactonyl esters (such as phthalidyl and thiophthalidyl esters), lower alkoxyacyloxyalkyl esters (such as methoxycarbonyl-oxymethyl, ethoxycarbonyloxyethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, α-amino acid amides, alkoxyacyl amides, and alkylaminoalkylcarbonyl amides. Examples of biohydrolyzable carbamates include, but are not limited to, lower alkylamines, substituted ethylenediamines, amino acids, hydroxyalkylamines, heterocyclic and heteroaromatic amines, and polyether amines.

As used herein, and unless otherwise specified, the term “stereoisomer” encompasses all enantiomerically/stereomerically pure and enantiomerically/stereomerically enriched compounds of this invention.

As used herein, and unless otherwise indicated, the term “stereomerically pure” or “enantiomerically pure” means that a compound comprises one stereoisomer and is substantially free of its counter stereoisomer or enantiomer. For example, a compound is stereomerically or enantiomerically pure when the compound contains 80%, 90%, or 95% or more of one stereoisomer and 20%, 10%, or 5% or less of the counter stereoisomer. In certain cases, a compound of the invention is considered optically active or stereomerically/enantiomerically pure (i.e., substantially the R-form or substantially the S-form) with respect to a chiral center when the compound is about 80% ee (enantiomeric excess) or greater, preferably, equal to or greater than 90% ee with respect to a particular chiral center, and more preferably 95% ee with respect to a particular chiral center.

As used herein, and unless otherwise indicated, the term “stereomerically enriched” or “enantiomerically enriched” encompasses racemic mixtures as well as other mixtures of stereoisomers of compounds of this invention (e.g., R/S=30/70, 35/65, 40/60, 45/55, 55/45, 60/40, 65/35 and 70/30).Various immunomodulatory compounds of the invention contain one or more chiral centers, and can exist as racemic mixtures of enantiomers or mixtures of diastereomers. This invention encompasses the use of stereomerically pure forms of such compounds, as well as the use of mixtures of those forms. For example, mixtures comprising equal or unequal amounts of the enantiomers of a particular immunomodulatory compounds of the invention may be used in methods and compositions of the invention. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, N.Y., 1962); and Wilen, S. H., Tables of Resolving Agents and Optical Resolutions p. 268 (E. L. Eliel, Ed., Univ. of Notre Dame Press, Notre Dame, Ind., 1972).

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

5.3. PHARMACEUTICAL COMPOSITIONS AND DOSAGE FORMS

Pharmaceutical compositions can be used in the preparation of individual, single unit dosage forms. Pharmaceutical compositions and dosage forms of the invention comprise an immunomodulatory compound of the invention, or a pharmaceutically acceptable salt, solvate, hydrate, stereoisomer, clathrate, or prodrug thereof. Pharmaceutical compositions and dosage forms of the invention can further comprise one or more excipients.

Pharmaceutical compositions and dosage forms of the invention can also comprise one or more additional active ingredients. Consequently, pharmaceutical compositions and dosage forms of the invention comprise the active ingredients disclosed herein (e.g., an immunomodulatory compound and a second active agent). Examples of optional second, or additional, active ingredients are disclosed herein (see, e.g., section 5.1).

Single unit dosage forms of the invention are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, this invention encompasses pharmaceutical compositions and dosage forms that contain little, if any, lactose other mono-or disaccharides. As used herein, the term “lactose-free” means that the amount of lactose present, if any, is insufficient to substantially increase the degradation rate of an active ingredient.

Lactose-free compositions of the invention can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms of the invention comprise an immunomodulatory compound of the invention or a pharmaceutically acceptable salt, solvate, hydrate, stereoisomer, clathrate, or prodrug thereof in an amount of from about 0.10 to about 150 mg. Typical dosage forms comprise an immunomodulatory compound of the invention or a pharmaceutically acceptable salt, solvate, hydrate, stereoisomer, clathrate, or prodrug thereof in an amount of about 0.1, 1, 2, 5, 7.5, 10, 12.5, 15, 17.5, 20, 25, 50, 100, 150 or 200 mg. In a particular embodiment, a preferred dosage form comprises 4-(amino)-2-(2,6-dioxo(3-piperidyl))-isoindoline-1,3-dione (i.e., a-(3-aminophthalimido) glutarimide) in an amount of about 1, 2, 5, 10, 25 or 50 mg. In a specific embodiment, a preferred dosage form comprises 3-(4-amino-1-oxo-1,3-dihydro-isoindol-2-yl)-piperidine-2,6-dione in an amount of about 5, 10, 25 or 50 mg. Typical dosage forms comprise the second active ingredient in an amount of 1 to about 1000 mg, from about 5 to about 500 mg, from about 10 to about 350 mg, or from about 50 to about 200 mg. Of course, the specific amount of the anti-cancer drug will depend on the specific agent used, the type of cancer being treated or managed, and the amount(s) of an immunomodulatory compound of the invention and any optional additional active agents concurrently administered to the patient.

Where one or more compounds of the invention are administered to an individual with a cytokine, the cytokine may be used in any pharmaceutically-acceptable dosage form, as described elsewhere herein, or acceptable concentration. Typically, for example, Neupogen is administered as an injectable bolus at a dose of from about 4 to about 8 micrograms/kg/day until a neutrophil count of 10,000/mm³ is reached. Ancestim (recombinant methionyl human stem cell factor) is typically administered via subcutaneous injection (but not intravenous injection) from 1-20 micrograms/kg/day for 9-12 days; recombinant human stem cell factor may be administered at a similar dosage. Sargramostim is typically administered at a dosage of up to about 250 micrograms/m²/day intravenously or subcutaneously, up to the time when white blood cell counts exceed about 50,000/mm³. Pegfilgrastim (Neulasta™) is typically administered at a dosage of about 6 milligrams subcutaneous, as needed. Appropriate dosages of cytokines that affect the number of white blood cells in the blood may be determined on a per-patient basis by determining the number of the particular white blood cell population, or the number of total white blood cells. Recombinant IL-3 may be obtained from, e.g., R&D Systems, Inc. (Minneapolis, Minn.). Recombinant IL-3 has an ED₅₀ of about 0.1 to about 0.4 ng/ml in vitro, and may be used at an equivalent concentration in vivo. Recombinant human stem cell factor (SCF) may be obtained from, e.g., BioSource International (Camarillo, Calif.). Recombinant SCF has an ED₅₀ of about 2 to about 5 ng/ml in vitro, and may be used at an equivalent concentration in vivo. Recombinant human Fms-Like Tyrosine Kinase-3 Ligand (Flt-3L) may be obtained from, e.g., ProSpec-Tany TechnoGene LTD (Rehovot, Israel) or U.S. Biological (Swampscott, Mass.). Recombinant human Flt-3L has an ED₅₀ of about 1 to about 10 ng/ml in vitro, and may be used at an equivalent concentration in vivo. Actual working concentrations of any of the foregoing may be determined on an individual basis by determining changes over time in the number of white blood cells or red blood cells in a culture or in blood samples drawn from an individual, according to practices known in the art. Differentiation of CD34+ cells along an erythroid pathway, and expression of fetal hemoglobin genes, can be assessed using known techniques (e.g., PCR-mediated or antibody-mediated detection of fetal hemoglobin transcripts or fetal hemoglobin).

Erythropoietin (e.g., Epogen™) is typically administered at a dosage of from about 12.5 U/kg to 525 U/kg, frequently about 100 U/kg or less, intravenously or subcutaneously. A variant of erythropoietin, Aranesp™, is typically administered at a similar dosage. For erythropoietin and erythropoietin analogs, the appropriate dosage is the dosage that results in a hematocrit of between about 10 g/dL and about 12 g/dL, and which avoids a rise of more than 1.0 g /dL in any 2-week period.

5.3.1. Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

A preferred solid oral dosage form of the invention comprises an immunomodulatory compound of the invention, anhydrous lactose, microcrystalline cellulose, polyvinylpyrrolidone, stearic acid, colloidal anhydrous silica, and gelatin.

### 5.3.2. Delayed Release Dosage Forms

Active ingredients of the invention can be administered by controlled release means or by delivery devices that are well known to those of ordinary skill in the art. Examples include, but are not limited to, those described in U.S. Pat. Nos.: 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

### 5.3.3. Parenteral Dosage Forms

Parenteral dosage forms can be administered to patients by various routes including, but not limited to, subcutaneous, intravenous (including bolus injection), intramuscular, and intraarterial. Because their administration typically bypasses patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention. For example, cyclodextrin and its derivatives can be used to increase the solubility of an immunomodulatory compound of the invention and its derivatives. See, e.g., U.S. Pat. No. 5,134,127, which is incorporated herein by reference.

5.3.4. Topical and Mucosal Doage Forms

Topical and mucosal dosage forms of the invention include, but are not limited to, sprays, aerosols, solutions, emulsions, suspensions, eye drops or other ophthalmic preparations, or other forms known to one of skill in the art. See, e.g., Remington's Pharmaceutical Sciences, 16^{th} and 18^{th} eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16^{th} and 18^{th} eds., Mack Publishing, Easton Pa. (1980 & 1990).

The pH of a pharmaceutical composition or dosage form may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### 5.3.5. Kits

Typically, active ingredients of the invention are preferably administered to a patient at the same time and by different routes of administration, but may be administered at different times or by the same route of administration. This invention therefore encompasses kits which, when used by the medical practitioner, can simplify the administration of appropriate amounts of active ingredients to a patient.

A typical kit of the invention comprises a dosage form of an immunomodulatory compound of the invention, or a pharmaceutically acceptable salt, solvate, hydrate, stereoisomer, prodrug, or clathrate thereof. Preferably, the immunomodulatory compound provided with the kit is 4-(Amino)-2-(2,6-dioxo(3-piperidyl))-isoindoline-1,3-dione or 3-(4-amino-1-oxo-1,3-dihydroisoindol-2-yl)-piperidine-2,6-dione or a compound having the formula:

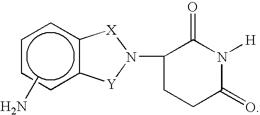

Kits encompassed by this invention can further comprise cytokines or cytokine derivatives such as G-CSF, GM-CSF, Epo, Flt-3L, SCF, IFN, IL2, IL8, IL18, etc., and/or other compounds, including but not limited to any other compound known or suspected to have a beneficial effect on anemia or a hemoglobinopathy, oblimersen (Genasense®), melphalan, topotecan, dacarbazine, irinotecan, taxotere, COX-2 inhibitor, pentoxifylline, ciprofloxacin, dexamethasone, Ara-C, vinorelbine, isotretinoin, 13 cis-retinoic acid, or a pharmacologically active mutant or derivative thereof, or a combination thereof. Other compounds that may be included in a kit include one or more of: a compound that induces fetal hemoglobin; a compound that relaxes blood vessels; a compound that when covalently bound to hemoglobin S reduces the self-aggregation of hemoglobin S; a compound that is a Gardos channel antagonist; and a compound that reduces red blood cell adhesion. In a more specific embodiment, said second compound is hydroxyurea, a guanidino derivative, nitrous oxide, butyrate or a butyrate derivative, an aldehyde or an aldehyde derivative, a plant extract having antisickling activity (e.g., NIPRISAN™ (HEMOXIN™)), clotrimazole, a derivative of triarylmethane, a monoclonal antibody or a polyethylene glycol derivative. Examples of the additional active ingredients include, but are not limited to, those disclosed herein (see, e.g., section 5.1).

Where some components of a course of treatment of a hemoglobinopathy are to be taken orally (e.g., immunomodulatory compounds, e.g., IMiDs™, e.g., 4-(Amino)-2-(2,6-dioxo(3-piperidyl))-isoindoline-1,3-dione or 3-(4-amino- 1-oxo-1,3-dihydroisoindol-2-yl)-piperidine-2,6-dione; extracts) and others are to be administered by another common route, e.g., intravenous or subcutaneous, a kit according to the invention can comprise components or compounds to be administered, other than the immunomodulatory compound(s) of the invention, for use as an adjunct to the immunomodulatory compounds.

Kits of the invention can further comprise devices that are used to administer the active ingredients. Examples of such devices include, but are not limited to, syringes, drip bags, patches, and inhalers.

Kits of the invention can further comprise cells or blood for transplantation as well as pharmaceutically acceptable vehicles that can be used to administer one or more active ingredients. For example, if an active ingredient is provided in a solid form that must. be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### 6. EXAMPLES

6.1. Example 1

Differentiation of Bone Marrow-Derived CD34⁺ hematopoietic progenitor cells to dendritic cells showing upregulated erythroid-specific genes

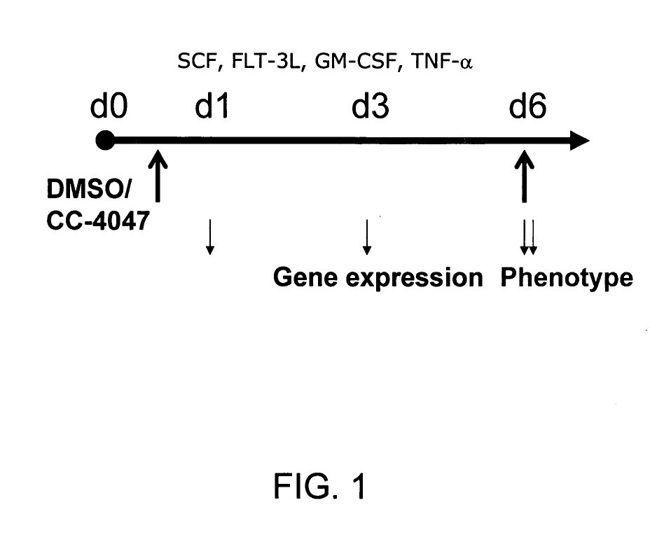

BM-CD34⁺ cells were obtained from Cambrex (East Rutherford, N.J.) and cultured in Iscove's MDM with BIT 95000 (StemCell Technologies, UK) in the presence of stem cell factor (SCF), Flt3-L, granulocyte macrophage-colony stimulating factor (GM-CSF) and TNFα for 6 days. To study the effect of 4-(Amino)-2-(2,6-dioxo(3-piperidyl))-isoindoline-1,3-dione on the generation of dendritic cells, CD34⁺ progenitor cells were cultured with or without 4-(Amino)-2-(2,6-dioxo(3-piperidyl))-isoindoline-1,3-dione for a period of 6 days. Phenotypic characterization of the cells for erythroid markers (CD36, CD71, glycophorin A and fetal hemoglobin) was established by flow cytometry after six days of culture. Gene expression was monitored by microarray analysis at day 1, day 3 and day 6 of CD34⁺ differentiation (FIG. 1).

RNA purification and microarray analysis. Total RNA was isolated from CD34⁺ cells using RNAeasy (Qiagen). Affymetrix U133A gene chips were used for gene expression analysis. Briefly, double-stranded cDNA was synthesized using 5 μg of total RNA. Biotin-labeled cRNA was synthesized using MessageAmp aRNA kit (Ambion), 15 μg of cRNA was fragmented and hybridized to each array. The above procedures were done twice for each RNA sample to obtain replicate biotin-labeled probes. The results from the replicate chips were averaged for calculation of the fold differences.

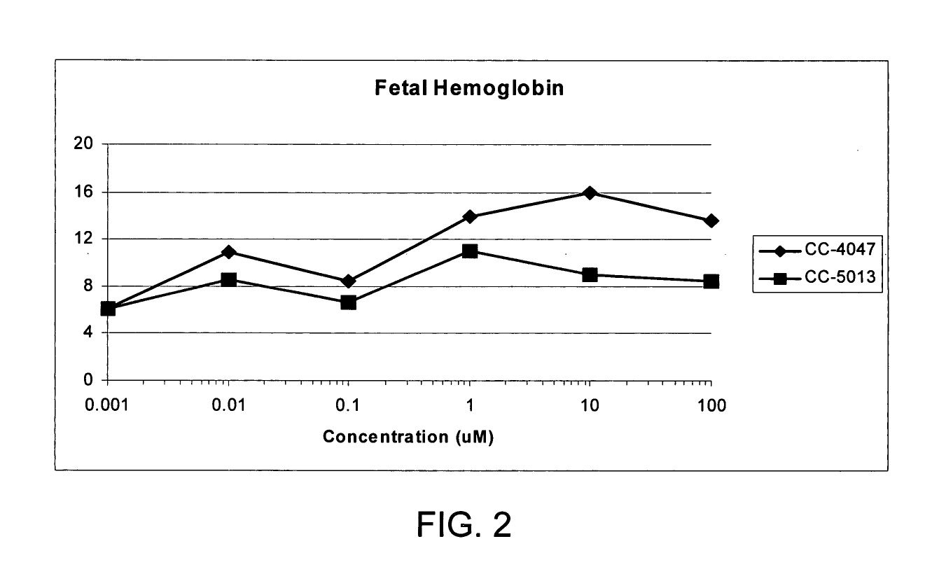

Results. 4-(Amino)-2-(2,6-dioxo(3-piperidyl))-isoindoline-1,3-dione treatment upregulated the gene expression profile of erythroid-specific genes during CD34⁺ differentiation in the presence of SCF, Flt3-L, GM-CSF and TNFα. Importantly, 4-(Amino)-2-(2,6-dioxo(3-piperidyl))-isoindoline-1,3-dione increased fetal hemoglobin gene expression upon CD34⁺ cell differentiation, with a specific increase of embryonic hemoglobin ε of 18-fold at day 6, and an increase of hemoglobin γ of seven-fold at day 6 (FIG. 2).

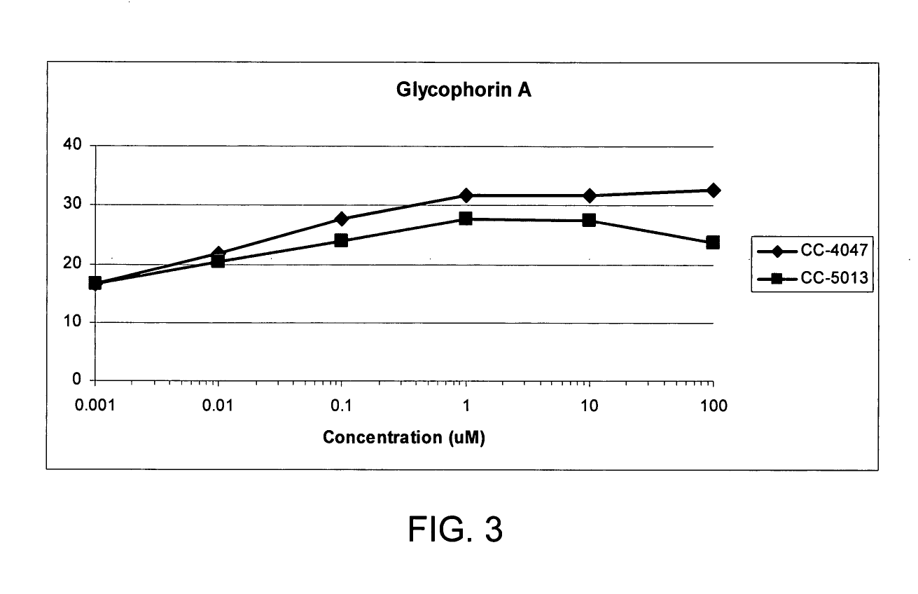

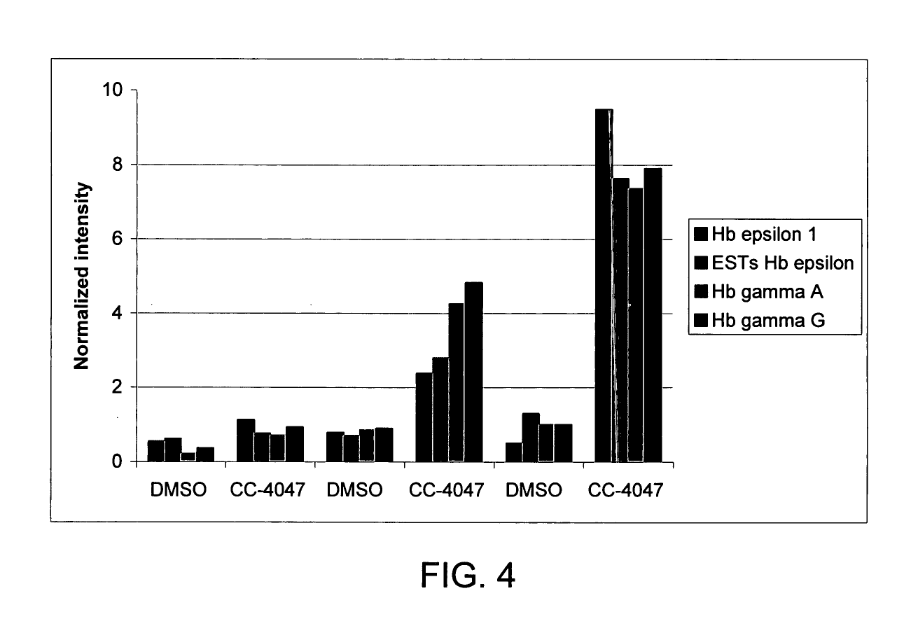

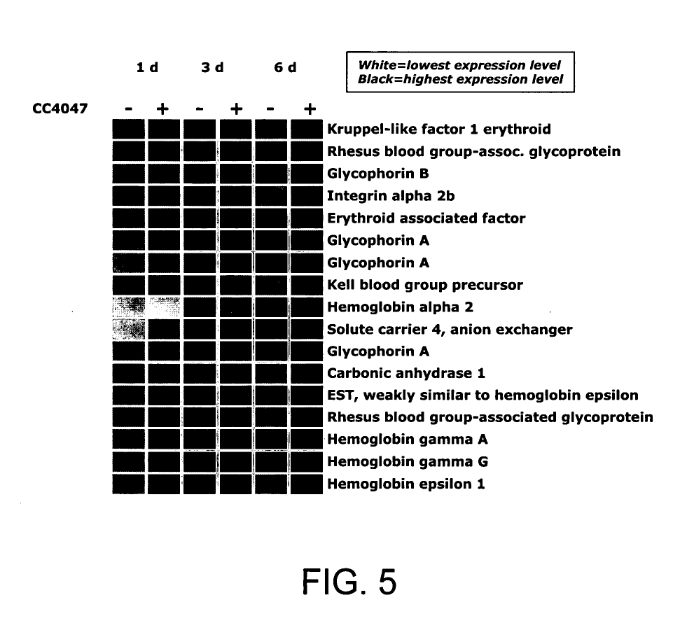

Phenotypic characterization by flow cytometry of CD34⁺ cells differentiated in the presence or absence of 4-(Amino)-2-(2,6-dioxo(3-piperidyl))-isoindoline-1,3-dione or 3-(4-amino-1-oxo-1,3-dihydroisoindol-2-yl)-piperidine-2,6-dione showed modulation of erythroid and hemoglobin markers. The expression of glycophorin A (FIG. 3) and fetal hemoglobin (FIG. 4) increased in a dose-dependent manner. 4-(Amino)-2-(2,6-dioxo(3-piperidyl))-isoindoline-1,3-dione also induced other erythroid-specific genes (FIG. 5). The expression of genes encoding glycophorin B, rhesus blood group associated glycoprotein, Kell blood group precursor, EDRF/AHSP (alpha hemoglobin stabilizing protein), and erythroid Kruppel-like transcription factor, each absolutely required for normal erythropoiesis, were also found to be upregulated in CD34⁺ cells differentiated in the presence of 4-(Amino)-2-(2,6-dioxo(3-piperidyl))-isoindoline-1,3-dione.

Many of the erythroid-specific genes increased by 4-(Amino)-2-(2,6-dioxo(3-piperidyl))-isoindoline-1,3-dione have a clear role in improving anemia. The increase in hemoglobin levels and alpha-hemoglobin stabilizing protein (AHSP) would both improve oxygen carrying capacity while protecting cells having excess alpha-hemoglobin levels, which can damage red blood cells. The IMiD effects on increasing erythropoiesis in general, and the genes noted above in particular, would be useful in overcoming the anemic effects of chemotherapy, as well as disease conditions in which low red blood cell count is a symptom or an effect of treatment.

It is anticipated that the effects of IMiDs™ will be synergistic to those of erythropoietin. IMiDs™ appear to induce the synthesis of early erythroid precursors, while erythropoietin is crucial for proliferation, survival and differentiation of the erythroid progenitors in the later stages of differentiation.

6.2. Example 2

Differentiation of CD34⁺ Cells to Erythroid Cells

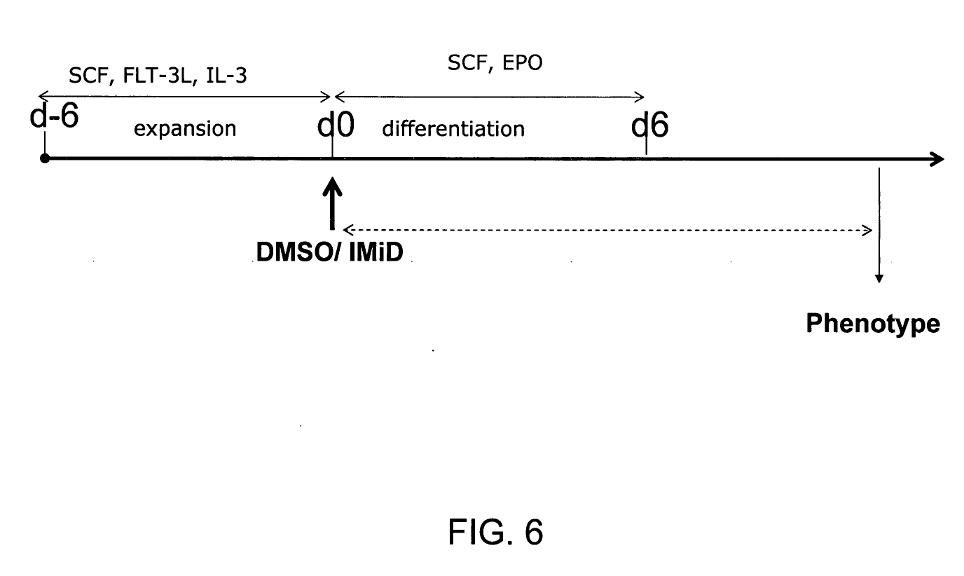

Differentiation of bone marrow (BM) CD34⁺ hematopoietic progenitor cells: BM-CD34⁺ progenitors were obtained from Cambrex and cultured in Iscove's MDM with BIT 95000 (serum substitute; StemCell technologies) in the presence of growth factors. During the first 6 days CD34⁺ cells were expanded with SCF (100 ng/ml), Flt3-L (100 ng/ml) and IL-3 (20 ng/ml), and then differentiated toward the erythroid lineage by culture in the presence of SCF (50 ng/ml), and Epo (2U or 4U/ml) for 6 days. To study the effect of IMiDs™, CD34⁺ progenitors cells were differentiated for a period of 6 days in the presence or absence of 4-(Amino)-2-(2,6-dioxo(3-piperidyl))-isoindoline-1,3-dione or 3-(4-amino-1-oxo-1,3-dihydroisoindol-2-yl)-piperidine-2,6-dione. (FIG. 6)

Flow cytometry: Surface antigen expression was analyzed by flow cytometry (FACScan, Coulter) after 6 days of culture. Cells were processed for double staining (30 min at 4° C.) at day 6 using FITC and PE conjugated monoclonal antibodies (mAbs). Antibodies used were: CD34-PE, CD36-FITC, CD71-FITC and Glycophorin A-PE, all from BD Pharmingen (San Diego, Calif.). After 6 days of culture, cells were washed with phosphate-buffered saline (PBS), fixed with 2% paraformaldehyde, permeabilized with cytopermeafix (BD Pharmingen) and stained with HbF-PE (BD Pharmingen, San Diego, Calif.), Hbε-FITC (Cortex Biochem, San Leandro, Calif.) mAbs and HbA-FITC (Perkin Elmer) and analyzed by flow cytometry (FACScan, Coulter or FCASAria, BD Pharmingen).

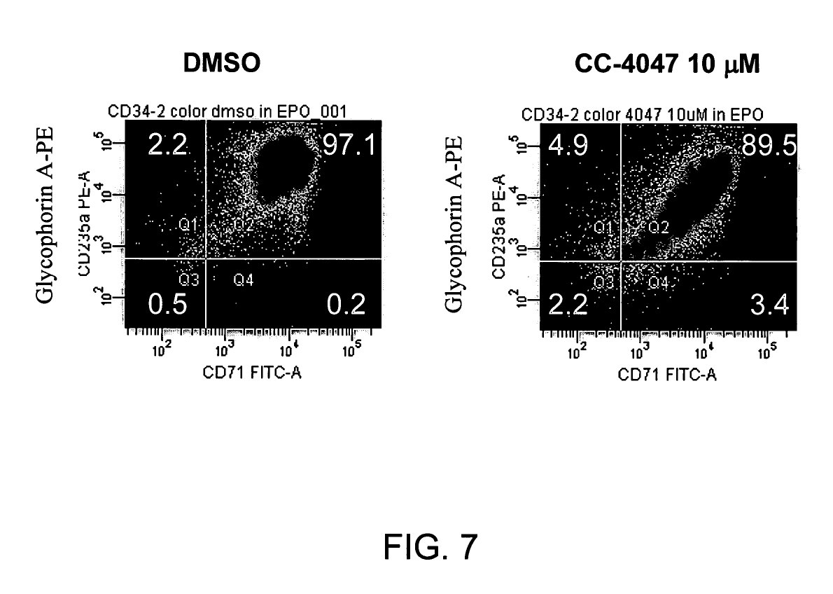

Results: IMiDs™ 4-(Amino)-2-(2,6-dioxo(3-piperidyl))-isoindoline-1,3-dione and 3-(4-amino-1-oxo-1,3-dihydroisoindol-2-yl)-piperidine-2,6-dione are potent inducers of hemoglobin F in erythroid precursors. CD34⁺ progenitors cells were first expanded with a combination of growth factors (SCF, Flt3-L and IL-3) for 6 days. After expansion CD34 cells were differentiated toward the erythroid lineage with SCF and Epo for 6 days, in the presence or absence of IMiDs™ (FIG. 6). The differentiation of CD34⁺ progenitors cells in the presence of SCF and Epo was monitored by the expression of characteristic erythroid surface markers: Glycophorin A (CD235) and the transferrin receptor (CD71) (FIG. 7). The erythroid phenotype was present when CD34⁺ cells were differentiated with or without IMiDs™. Interestingly, expression of Glycophorin A was lower in IMiD™-treated cells, while expression of CD71 was maintained at a high level in both conditions.

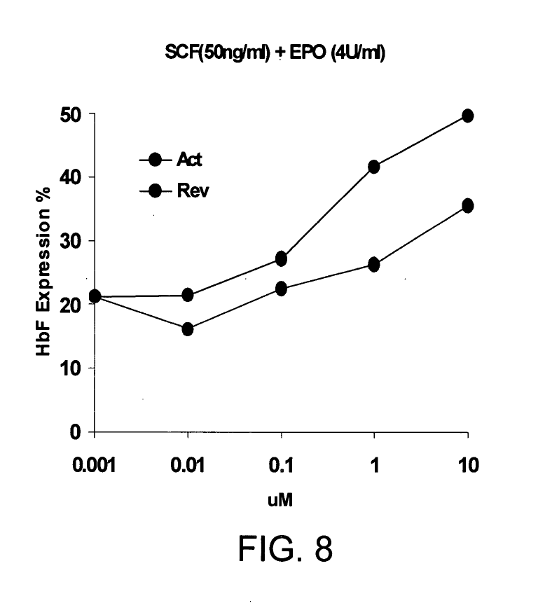

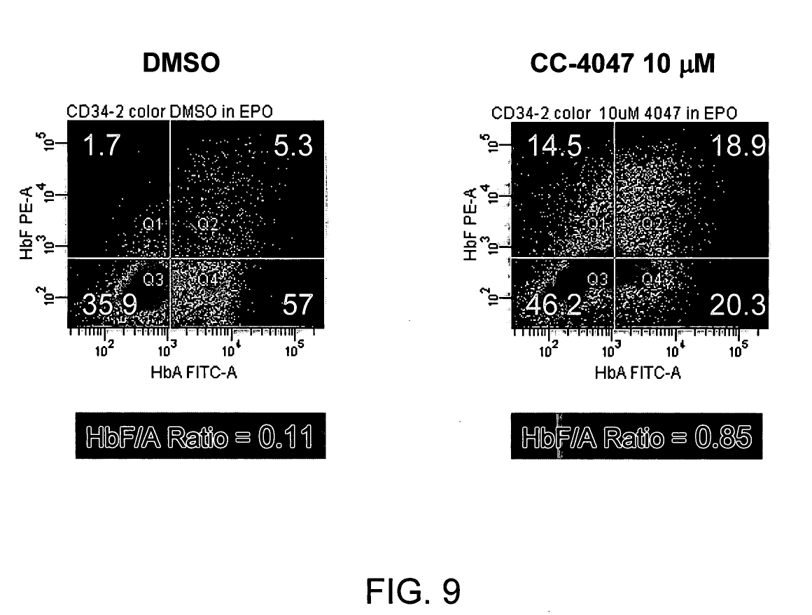

The percentage of cells expressing fetal hemoglobin was monitored by flow cytometry after 6 days of culture with SCF and Epo. The expression of fetal hemoglobin was increased in a dose-dependent manner by IMiDs™ (FIG. 8). Importantly, the increase in fetal hemoglobin (HbF) was associated with a decrease in adult hemoglobin (HbA). The ratio of HbF/HbA increased in the presence of IMiDs™. (FIG. 9)

In addition to phenotypic maturation, hemoglobin quantitation, the proliferation status of the cells was also measured. Cells counts were performed after 6 days of culture with SCF and Epo. The total cells counts was increased in the presence of IMiDs™ and correlated well with the developmental stage of the population (i.e., less mature).

6.3. Example 3

IMIDs act synergistically with current fetal hemoglobin approved therapies

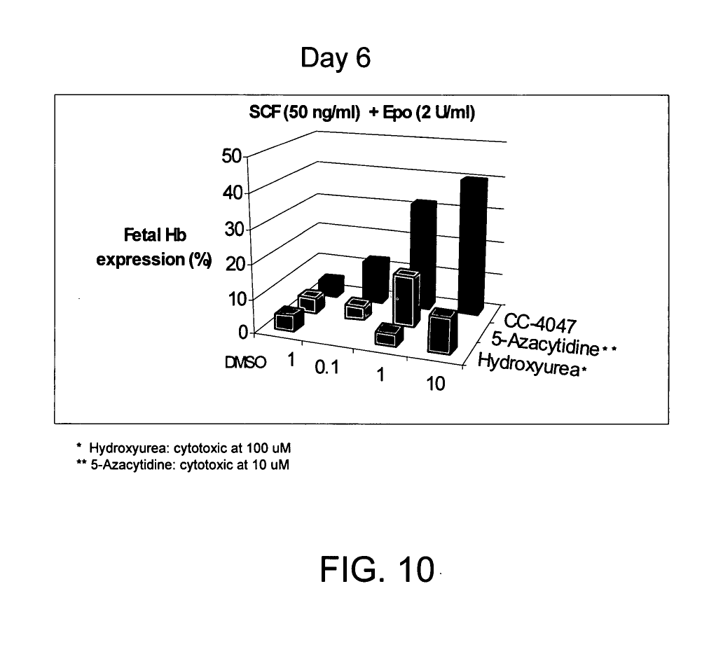

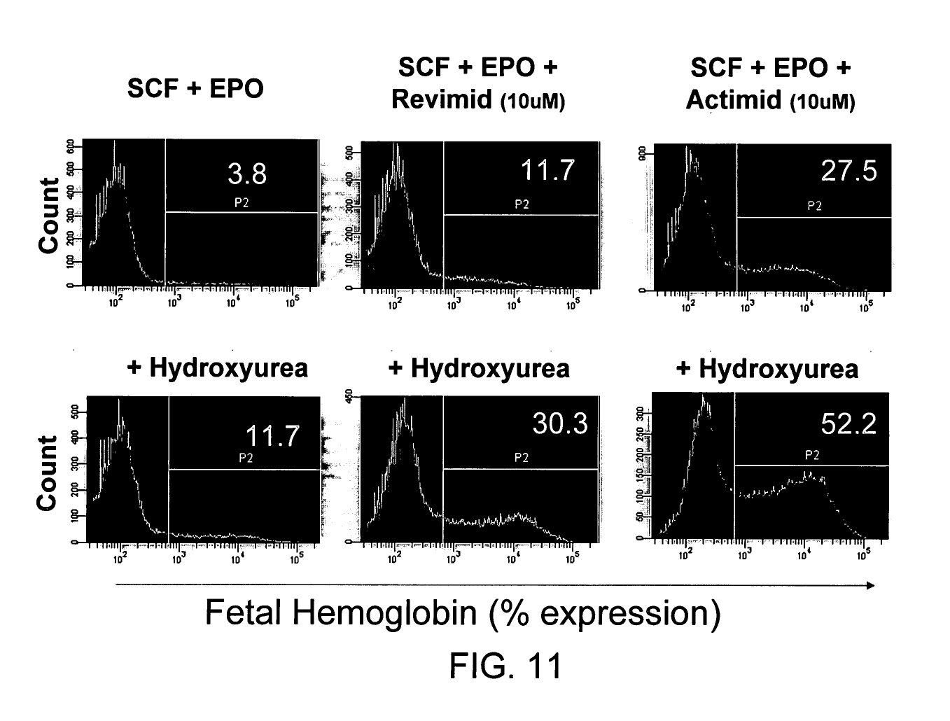

As previously, CD34⁺ progenitor cells were first expanded with a combination of growth factors (SCF, Flt3-L and IL-3) for 6 days, and erythroid differentiation was then induced with SCF and Epo for 6 days. During the erythroid differentiation period CD34⁺ cells were cultured in the presence or absence of IMiDs™, alone or in combination with either hydroxyurea and 5-azacytidine, in order to compare the effect of IMiDs™ to these two known inducers of fetal hemoglobin synthesis. On day 6 of differentiation, the hemoglobin content of the cells was measured by flow cytometry. Hydroxyurea and 5-azacytidine increased fetal hemoglobin expression as reported (FIG. 10). The induction of fetal hemoglobin production was, however, more pronounced with IMiD in comparison to hydroxyurea or 5-azacytidine, with a 10 fold induction in the presence of 10 μM of 4-(Amino)-2-(2,6-dioxo(3-piperidyl))-isoindoline-1,3-dione. Interestingly, 4-(Amino)-2-(2,6-dioxo(3-piperidyl))-isoindoline-1,3-dione showed a striking synergy in combination with hydroxyurea, resulting in a striking reactivation of fetal hemoglobin (FIG. 11).

6.4. Example 4

EPO+IMIDs cause an increase in stat4 phosphorylation

To further characterize the synergy of Epo and IMiDs™ on erythroid cells, we have performed signaling experiments in a UT-7 cell line, in particular, to determine the effect if IMiDs on the expression of STAT5, which is known to be activated upon the binding Epo to the erythropoietin receptor (EpoR). UT-7 is a human leukemia cell line absolutely dependent upon erythropoietin for proliferation, and was isolated from a patient with acute myeloid leukemia (AML M7). The level of EpoR expression in these cells is around 60%.

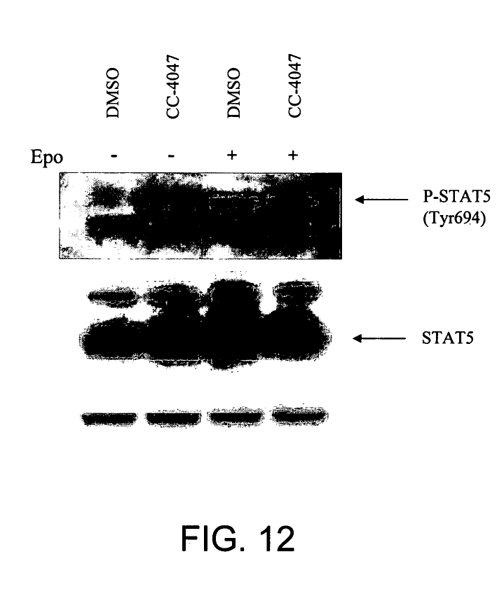

To study the role of IMiDs™ in Epo signaling, we stimulated UT-7 cells with Epo in the presence or absence of 4-(Amino)-2-(2,6-dioxo(3-piperidyl))-isoindoline- 1,3-dione as follows. UT-7 cells were expended in RPMI medium with 10% FBS and GM-CSF (5 ng/ml). The cells were serum and growth factor starved overnight, then pre-incubated for 45 minutes with 10 μM of 4-(Amino)-2-(2,6-dioxo(3-piperidyl))-isoindoline-1,3-dione or DMSO control, and stimulated with Epo (10 U/ml) for 10 minutes. 4-(Amino)-2-(2,6-dioxo(3-piperidyl))-isoindoline-1,3-dione increased Epo-induced STAT5 (Tyr694) phosphorylation by 2 fold (FIG. 12). This effect was detected within 10 minutes of stimulation with Epo.

### 6.5. Example 5

Toxicology Studies

The effects of 3-(4-amino-1-oxo-1,3-dihydro-isoindol-2-yl)-piperidine-2,6-dione on cardiovascular and respiratory function are investigated in anesthetized dogs. Two groups of Beagle dogs (2/sex/group) are used. One group receives three doses of vehicle only and the other receives three ascending doses of 3-(4-amino-1-oxo-1,3-dihydro-isoindol-2-yl)-piperidine-2,6-dione (2, 10, and 20 mg/kg). In all cases, doses of 3-(4-amino-1-oxo-1,3-dihydro-isoindol-2-yl)-piperidine-2,6-dione or vehicle are successively administered via infusion through the jugular vein separated by intervals of at least 30 minutes.

The cardiovascular and respiratory changes induced by 3-4-amino-1-oxo-1,3-dihydro-isoindol-2-yl)-piperidine-2,6-dione are minimal at all doses when compared to the vehicle control group. The only statistically significant difference between the vehicle and treatment groups is a small increase in arterial blood pressure (from 94 mmHg to 101 mmHg) following administration of the low dose of 3-(4-amino-1-oxo-1,3-dihydro-isoindol-2-yl)-piperidine-2,6-dione. This effect lasts approximately 15 minutes and is not seen at higher doses. Deviations in femoral blood flow, respiratory parameters, and Qtc interval are common to both the control and treated groups and are not considered treatment-related.

The embodiments of the invention described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

### 7. REFERENCES

All references cited herein are incorporated herein by reference in their entirety and for all purposes to the same extent as if each individual publication, patent or patent application was specifically and individually indicated to be incorporated by reference in its entirety for all purposes. The citation of any publication is for its disclosure prior to the filing date and should not be construed as an admission that the present invention is not entitled to antedate such publication by virtue of prior invention.

1. A method of treating an individual having a hemoglobinopathy or an anemia, said method comprising administering to said individual an immunomodulatory compound in an amount and for a time sufficient to reduce one or more symptoms of said hemoglobinopathy.

2. The method of claim 1, wherein said hemoglobinopathy is sickle cell anemia or thalassemia.

3. The method of claim 1, where said anemia is an anemia induced or related to the administration of a chemotherapy or drug.

4. The method of claim 1, wherein said individual is administered a pharmaceutically acceptable salt, solvate, hydrate, stereoisomer, clathrate or prodrug of an immunomodulatory compound.

5. The method of claim 4, wherein said immunomodulatory compound is an amino-substituted thalidomide.

6. The method of claim 4, wherein said immunomodulatory compound is α-(3-aminophthalimido) glutarimide; an analog or prodrug of α-(3-aminophthalimido) glutarimide; 3-(4′aminoisoindoline-1′-one)-1-piperidine-2,6-dione; an analog or prodrug of 3-(4′aminoisoindoline-1′-one)-1-piperidine-2,6-dione; or a compound of the formula

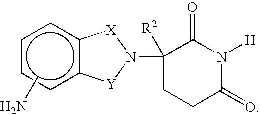

7. The method of claim 4, wherein said immunomodulatory compound is 1-oxo-2-(2,6-dioxopiperidin-3-yl)-5-aminoisoindoline; 1-oxo-2-(2,6-dioxopiperidin-3-yl)-4-aminoisoindoline; 1-oxo-2-(2,6-dioxopiperidin-3-yl)-6-aminoisoindoline; 1-oxo-2-(2,6-dioxopiperidin-3-yl)-5-aminoisoindoline 1,3 dioxo-2-(2,6-dioxopiperidin-3-yl)-4-aminoisoindoline; or 1,3 dioxo-2-(2,6-dioxopiperidin-3-yl)-5-aminoisoindoline.

8. The method of claim 1, additionally comprising treating said individual with a second compound, wherein said second compound is a compound that induces fetal hemoglobin, a compound that relaxes blood vessels, a compound that when covalently bound to hemoglobin S reduces the self-aggregation of hemoglobin S, a compound that is a Gardos channel antagonist, or a compound that reduces red blood cell adhesion.

9. The method of claim 8, wherein said second compound is hydroxyurea, a guanidino derivative, nitrous oxide, butyrate or a butyrate derivative, an aldehyde or an aldehyde derivative, a plant extract having antisickling activity, clotrimazole, a derivative of triarylmethane, a monoclonal antibody or a polyethylene glycol derivative.

10. The method of claim 1, additionally comprising treating said individual with at least one cytokine.

11. The method of claim 10, wherein said at least one cytokine is erythropoietin (Epo); SCF; GM-CSF; Flt-3L; TNFα; IL-3; or any combination thereof.

12. The method of claim 10, wherein said individual is treated with Epo and SCF.

13. The method of claim 1 wherein said individual is a mammal.

14. The method of claim 13 wherein said individual is a human.

15. The method of claim 10, wherein said treating comprises treating said individual with a combination of stem cell factor (SCF), Flt-3L and IL-3, and subsequently treating sand individual with a combination of SCF and erythropoietin, wherein said treating is sufficient to cause a detectable increase in the expression of at least one fetal hemoglobin gene.

16. A method of modulating the differentiation of a CD34⁺ stem or precursor cell to an erythroid lineage comprising differentiating said cell under suitable conditions and in the presence of an immunomodulatory compound.

17. The method of claim 16, wherein said differentiating is done in the presence of a pharmaceutically acceptable salt, solvate, hydrate, stereoisomer, clathrate or prodrug of an immunomodulatory compound.

18. The method of claim 17, wherein said immunomodulatory compound is an amino-substituted thalidomide.

19. The method of claim 17, wherein said immunomodulatory compound is α-(3-aminophthalimido) glutarimide; an analog or prodrug of α-(3-aminophthalimido) glutarimide; 3-(4′aminoisoindoline-1′-one)-1-piperidine-2,6-dione; an analog or prodrug of 3-(4′aminoisoindoline-1′-one)-1-piperidine-2,6-dione or a compound of the formula

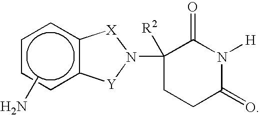

20. The method of claim 17, wherein said immunomodulatory compound is 1-oxo-2-(2,6-dioxopiperidin-3-yl)-5-aminoisoindoline; 1-oxo-2-(2,6-dioxopiperidin-3-yl)-4-aminoisoindoline; 1-oxo-2-(2,6-dioxopiperidin-3-yl)-6-aminoisoindoline; 1-oxo-2-(2,6-dioxopiperidin-3-yl)-5-aminoisoindoline; 1,3 dioxo-2-(2,6-dioxopiperidin-3-yl)-4-aminoisoindoline; or 1,3 dioxo-2-(2,6-dioxopiperidin-3-yl)-5-aminoisoindoline.

21. The method of claim 16, wherein said CD34⁺ stem or precursor cell is a cell in vitro.

22. The method of claim 16, wherein said CD34⁺ stem or precursor cell is a cell in vivo.

23. The method of claim 16, additionally comprising contacting said cell with at least one cytokine.

24. The method of claim 23, wherein said at least one cytokine is erythropoietin (Epo); SCF; GM-CSF; Flt-3L; TNF-α; IL-3; or any combination thereof.

25. A pharmaceutical composition comprising in a pharmaceutically-acceptable carrier a first immunomodulatory compound and a second compound, wherein said second compound is a compound that induces fetal hemoglobin, a compound that relaxes blood vessels, a compound that when covalently bound to hemoglobin S reduces the self-aggregation of hemoglobin S, a compound that is a Gardos channel antagonist, or a compound that reduces red blood cell adhesion.

26. The method of claim 25, wherein said second compound is hydroxyurea, a guanidino derivative, nitrous oxide, butyrate or a butyrate derivative, an aldehyde or an aldehyde derivative, a plant extract having antisickling activity, clotrimazole, a derivative of triarylmethane, a monoclonal antibody or a polyethylene glycol derivative.

27. A pharmaceutical composition comprising in a pharmaceutically-acceptable carrier an immunomodulatory compound and at least one cytokine, wherein said at least one cytokine is erythropoietin (Epo); SCF; GM-CSF; Flt-3L; TNF-α; IL-3; or any combination thereof.

28. A method of treating an individual having a hemoglobinopathy or anemia, said method comprising administering to said individual a compound in an amount and for a time sufficient to cause a detectable increase in the level of alpha hemoglobin stabilizing protein (AHSP).

29. The method of claim 28, wherein said compound is an immunomodulatory compound.

30. The method of claim 29, wherein said immunomodulatory compound is a-(3-aminophthalimido) glutarimide or 3-(4′aminoisoindoline- 1′-one)-1-piperidine-2,6-dione.
